(19) United States
(12) Patent Application Publication
(10) Pub. No.: US 2022/0091673 A1
(43) Pub. Date: Mar. 24, 2022

(54) OPERATOR STATE DETERMINING SYSTEM

(71) Applicant: Wacom Co., Ltd., Saitama (JP)

(72) Inventors: Hideki FUJIMAKI, Saitama (JP); Tsukasa NOMI, Saitama (JP)

(21) Appl. No.: 17/542,182

(22) Filed: Dec. 3, 2021

Related U.S. Application Data
(63) Continuation of application No. PCT/JP2020/018596, filed on May 8, 2020.

(30) Foreign Application Priority Data
Jun. 6, 2019 (JP) 2019-105778

(51) Int. Cl. G06F 3/01; G06F 3/0354

(52) U.S. Cl. CPC G06F 3/015; G06F 2203/011; G06F 3/03545

(57) ABSTRACT

An operator state determining system includes an information collecting device which, in operation, collects information used to determine whether an operator who operates an operation target device is in an inappropriate state for an operation, and a determining device which, in operation, determines whether the operator is in the inappropriate state for the operation. The information collecting device includes a biological information obtaining device which, in operation, obtains biological information of the operator, a writing trajectory information obtaining device which, in operation, obtains writing trajectory information corresponding to writing input by the operator, and a transmission circuit which, in operation, transmits the biological information and the writing trajectory information to the determining device. The determining device includes a state determining circuit which, in operation, determines whether the operator is in the inappropriate state for the operation based on the biological information and the writing trajectory information that are received.

### BACKGROUND

### Technical Field

The present disclosure relates to an operator state determining system that determines whether an operator (for example, a driver) operating (for example, driving) an operation target device (for example, an automobile) is in an inappropriate state for operation.

### Background Art

Safety comes first in driving an automobile because a traffic accident during the driving of the automobile, for example, causes a situation affecting human life in a worst case. When the driving of the automobile is performed for a long time, there is a fear that physical and mental fatigue may accumulate in the driver, reaction speed in driving operation may decrease, and a probability of occurrence of an accident may increase. In addition, an abrupt change in physical condition (condition of the body) of the driver driving the automobile may also increase the probability of occurrence of an accident.

Accordingly, a company that uses automobiles for business, the automobiles being, for example, buses or taxis traveling while carrying passengers as service or tracks or the like transporting entrusted loads, pays attention to the health states and physical conditions of drivers, and makes efforts to be able to maintain safe driving.

For example, a proposal has been made to prevent an accident by having a driver wear a biosensor, collecting and monitoring biological information, for example the heartbeat, the body temperature, perspiration, or the like of the driver from the biosensor at a management center, and thereby recognizing a change in the physical condition of the driver (see, for example, Japanese Patent Laid-Open No. 2017-217333 (Patent Document 1) and the like).

It is well known, however, that as a cause of occurrence of a human-induced traffic accident, not only the physical condition of the driver but also a mood or an emotion as the mental state (mental action state) of the driver has an effect. For example, the driver is regarded as having a tendency to cause a traffic accident when the driver is in a mental state (emotional state) of being irritated or angered, for example. Then, it has also been reported that in actuality, traffic accidents based on the mental states of drivers are more likely to occur than traffic accidents caused by changes in the physical conditions of the drivers.

Although the emotional state of a driver may appear as biological information, it is very difficult to grasp the mental state (emotional state) from the biological information. Therefore, because the mental state cannot be grasped by monitoring only the biological information of an operator such as a driver as in the past, it is difficult to avoid an unexpected situation such as a traffic accident effectively.

### BRIEF SUMMARY

It is an object of the present disclosure to provide an operator state determining system that can solve the above problems.

In order to solve the above problems, there is provided an operator state determining system including an information collecting device which, in operation, collects information used to determine whether an operator who operates an operation target device is in an inappropriate state for an operation, and a determining device which, in operation, determines whether the operator is in the inappropriate state for the operation. The information collecting device includes a biological information obtaining device which, in operation, obtains biological information of the operator, a writing trajectory information obtaining device which, in operation, receives writing input by the operator, and obtains writing trajectory information corresponding to the writing input, and a transmission circuit which, in operation, transmits the biological information obtained by the biological information obtaining device and the writing trajectory information obtained by the writing trajectory information obtaining device to the determining device. The determining device includes a reception circuit which, in operation, receives the biological information and the writing trajectory information transmitted from the information collecting device, and a state determining circuit which, in operation, determines whether the operator is in the inappropriate state for the operation based on the biological information and the writing trajectory information received by the reception circuit.

The operator state determining system having the above-described configuration includes the writing trajectory information obtaining device that receives the writing input by the operator, and obtains the writing trajectory information corresponding to the writing input. A human is known to draw a writing trajectory corresponding to a mental state (emotional state) at the time when the human performs a writing input. For example, when the human is irritated or angered, the mental state (emotional state) appears in the writing trajectory. Hence, the mental state (emotional state) of the operator can be grasped from the writing trajectory information of the operator.

That is, the determining device can grasp a change in the physical condition of the operator from the biological information, and can grasp the mental state (emotional state) of the operator from the writing trajectory information. It is therefore possible to determine from grasping results of both whether the operator, for example, a driver is in an inappropriate state for operating, for example, driving the operation target device, for example, an automobile.

Hence, by using a result of the determination of the determining device, it is possible to issue an alarm to the operator, or perform stop control on the operation target device so as to disable operation of the operation target device.

### BRIEF DESCRIPTION OF THE DRAWINGS

FIG. 1 is a diagram of assistance in explaining an outline of an example of a configuration of an operator state determining system according to a first embodiment of the present disclosure;

FIG. 2 is a diagram of assistance in explaining an example of configuration of a tablet terminal and an electronic pen constituting the operator state determining system according to the first embodiment of the present disclosure;

FIGS. 3A, 3B, 3C, and 3D are an exploded perspective views of assistance in explaining an example of a configuration of the tablet terminal constituting the operator state determining system according to the first embodiment of the present disclosure;

FIG. 4 is a diagram depicting an example of an electric configuration of the example of configuration of the tablet terminal and the electronic pen constituting the operator state determining system according to the first embodiment of the present disclosure;

FIG. 5 is a diagram depicting an example of a configuration of a management server device constituting the operator state determining system according to the first embodiment of the present disclosure;

FIG. 6 is a diagram used to describe an example of a configuration of the management server device in the example of FIG. 5;

FIG. 7 is a diagram used to describe an example of a configuration of the management server device in the example of FIG. 5;

FIG. 8 is a diagram depicting an example of a configuration of an emotion server device constituting the operator state determining system according to the first embodiment;

FIG. 9 is a diagram used to describe an example of a configuration of the emotion server device in the example of FIG. 8;

FIG. 10 is a diagram used to describe an example of a configuration of the emotion server device in the example of FIG. 8;

FIG. 11 is a diagram depicting a part of a flowchart of assistance in explaining an example of processing operation of the example of configuration of the tablet terminal constituting the operator state determining system according to the first embodiment;

FIG. 12 is a diagram depicting a part of the flowchart of assistance in explaining the example of processing operation of the example of configuration of the tablet terminal constituting the operator state determining system according to the first embodiment;

FIG. 13 is a diagram depicting a part of a flowchart of assistance in explaining an example of processing operation of the example of configuration of the management server device in the example of FIG. 5;

FIG. 14 is a diagram depicting a part of the flowchart of assistance in explaining the example of processing operation of the example of configuration of the management server device in the example of FIG. 5;

FIG. 15 is a diagram of assistance in explaining an outline of an example of a configuration of a second embodiment of the operator state determining system according to the present disclosure;

FIG. 16 is a diagram depicting an example of a configuration of an automotive device constituting the operator state determining system according to the second embodiment; and

FIG. 17 is a diagram depicting a flowchart of assistance in explaining an example of processing operation of the example of configuration of the automotive device in the example of FIG. 16.

### DETAILED DESCRIPTION

Embodiments of an operator state determining system according to the present disclosure will hereinafter be described with reference to the drawings.

### First Embodiment

### <Outline of Operator State Determining System According to First Embodiment>

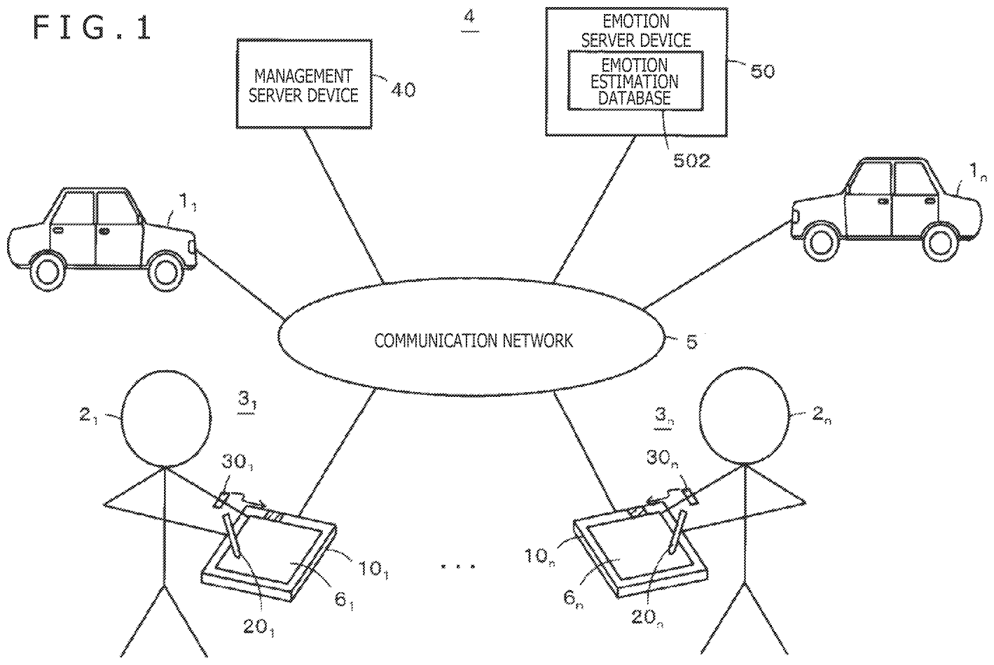

FIG. 1 is a diagram of assistance in explaining an outline of a first embodiment of an operator state determining system according to the present disclosure. The operator state determining system according to the first embodiment to be described in the following is an example in a case where an operation target device is an automobile used as a taxi, and a taxi company uses the operator state determining system in order to manage the states of taxi drivers (hereinafter referred to as drivers) as an example of operators.

As depicted in FIG. 1, the operator state determining system according to the present first embodiment is configured such that each of n (n=1, 2, . . . ) information collecting devices 3₁ to 3ₙ provided to n respective drivers 2₁ to 2ₙ as operators who operate n respective automobiles 1₁ to 1ₙ used as taxis and a determining device 4 constituted by a server device operated by the taxi company are connected to each other by wireless communication through a communication network 5. In the present example, each of the automobiles 1₁ to 1ₙ is also connected to the determining device 4 by wireless communication through the communication network 5, and is configured to be able to receive control from the determining device 4.

The operator state determining system according to the present first embodiment collects biological information such as the heartbeat, and body temperature of each of the drivers 2₁ to 2ₙ from biological information obtaining devices fitted to each of the drivers 2₁ to 2ₙ, collects writing trajectory information of an operation log as electronic data by utilizing a fact that each of the drivers 2₁ to 2ₙ generates the operation log, and determines and manages states of a physical condition and an emotion of each of the drivers 2₁ to 2ₙ on the basis of the collected biological information and the collected writing trajectory information.

In the present embodiment, each time each of the drivers 2₁ to 2ₙ provides a taxi service to a passenger, the driver performs writing input and recording of service contents in the operation log, and transmits the writing trajectory information of the operation log in which the service contents are recorded to the determining device 4 constituted by the server device operated by the taxi company together with the biological information of the driver.

The information collecting devices 3₁ to 3ₙ in the present example include respective tablet terminals 10₁ to 10ₙ assigned to the respective drivers 2₁, . . . , 2ₙ, respective pens 20₁, . . . , 20ₙ, and respective smart watches 30₁ to 30ₙ. The tablet terminals 10₁ to 10ₙ have a similar configuration. The electronic pens 20₁, . . . , 20ₙ also have a similar configuration. The smart watches 30₁ to 30ₙ also have a similar configuration. A pair of each of the tablet terminals 10₁ to 10ₙ and each of the electronic pens 20₁, . . . , 20ₙ includes functional means constituting an example of each of writing trajectory information obtaining means. In addition, each of the smart watches 30₁ to 30ₙ includes functional means constituting an example of biological information obtaining devices.

Incidentally, in the following description, when it is not necessary to distinguish each of then (n=1, 2, . . . ) automobiles 1₁ to 1ₙ, each of then drivers 2₁ to 2ₙ, and each of then information collecting devices 3₁ to 3ₙ, each of then automobiles 1₁ to 1ₙ, each of the n drivers 2₁ to 2ₙ, and each of then information collecting devices 3₁ to 3ₙ will be respectively described as an automobile 1, a driver 2, and an information collecting device 3 for the convenience of description. In addition, the n tablet terminals 10₁ to 10ₙ, the n electronic pens 20₁, . . . , 20ₙ, and the n smart watches 30₁ to 30ₙ have respective similar configurations. Thus, each of the n tablet terminals 10₁ to 10ₙ, each of the n electronic pens 20₁, . . . , 20ₙ, and each of then smart watches 30₁ to 30ₙ will be respectively described as a tablet terminal 10, an electronic pen 20, and a smart watch 30 for the convenience of description when it is not necessary to distinguish each.

The smart watch 30 is fitted to an arm of the driver 2 like a wristwatch. The smart watch 30 has a function of detecting the biological information of the driver 2 such as heartbeat, body temperature, perspiration, and blood pressure in the present example. The detected biological information includes information regarding a detection time. Then, the smart watch 30 in the present example has a short-range radio communication circuit of a Bluetooth (registered trademark) standard, for example, to transmit the detected biological information to the tablet terminal 10.

The tablet terminal 10 in the present example is provided with a wireless communication circuit of the Bluetooth (registered trademark) standard for communicating with the smart watch 30. The tablet terminal 10 receives and obtains the biological information of each driver 2 from the smart watch 30.

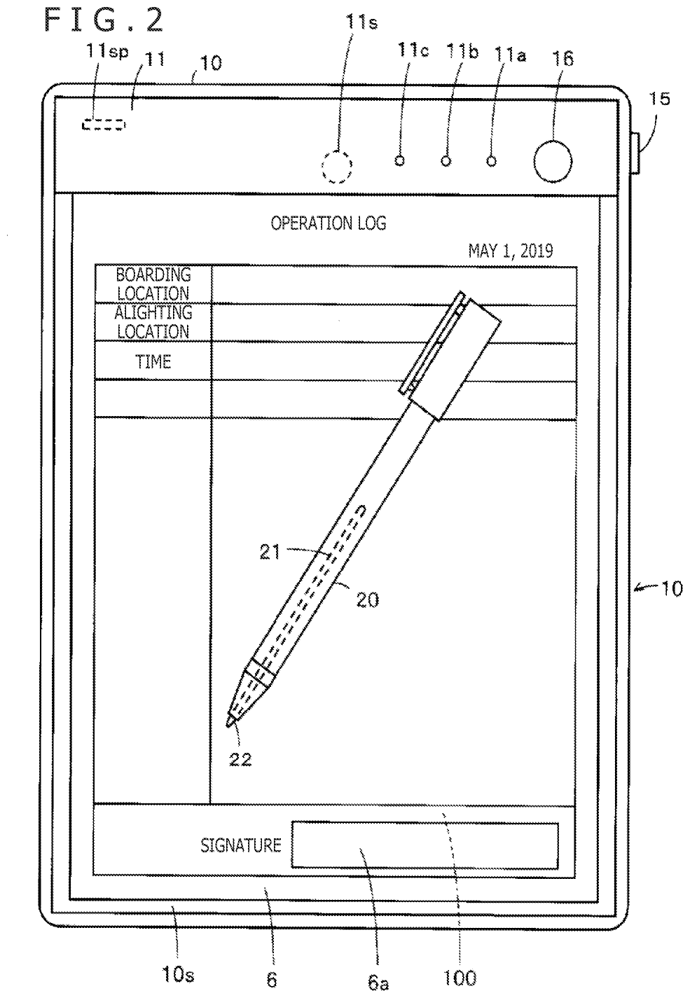

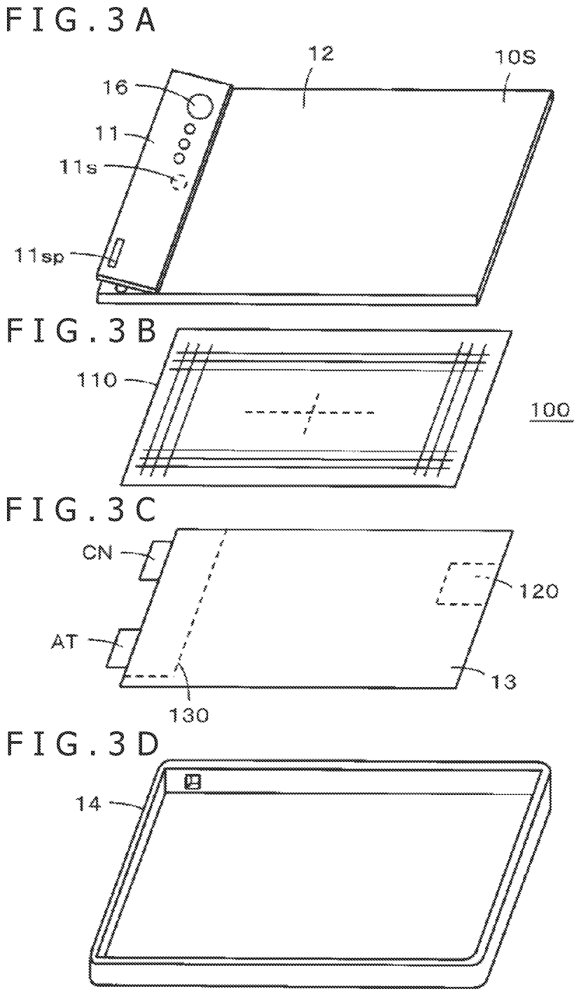

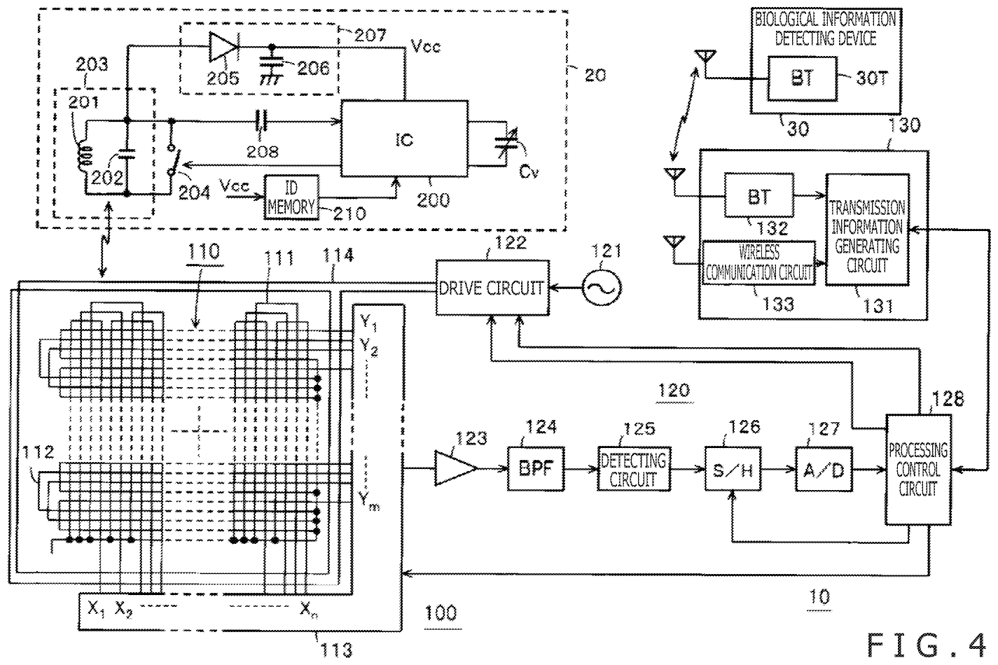

Then, the tablet terminal 10 in the present example includes a position detecting device 100 (see FIG. 2 and FIGS. 3A to 3D to be described later) that detects a position indicated by the electronic pen 20 by interacting with the electronic pen 20 by electromagnetic induction coupling.

In the present example, the driver 2 of a taxi generates an operation log by performing writing input to the tablet terminal 10 by using the electronic pen 20. The tablet terminal 10 detects the writing input by the electronic pen 20, and generates and obtains writing trajectory information of the operation log as electronic data.

Further, the tablet terminal 10 in the present example includes a wireless communication circuit that wirelessly communicates with the determining device 4 through the communication network 5. Then, as will be described later, the tablet terminal 10 adds identification information identifying the driver 2 to the obtained writing trajectory information of the operation log and the biological information of the driver 2 which biological information is obtained from the smart watch 30, and transmits resulting information to the determining device 4 through the communication network 5.

As depicted in FIG. 1, the determining device 4 in the present embodiment includes a management server device 40 and an emotion server device 50. The management server device 40 receives the writing trajectory information of the operation log and the biological information of each driver 2 from each tablet terminal 10.

As will be described later, the management server device 40 includes a biological information database. The management server device 40 checks the physical condition of each driver 2 by analyzing the received biological information of each driver 2 by using data stored in the biological information database, and determines whether the physical condition of each driver 2 is an inappropriate state with regard to the driving of the automobile 1. Then, the management server device 40 transmits an alarm to each driver 2 whose physical condition is determined to be an inappropriate state with regard to the driving of the automobile 1, and prompts the driver 2 to suspend or stop the driving of the respective automobile 1.

In addition, together with this alarm transmission, the management server device 40 sends control information for performing remote control so as to temporary stop or stop traveling forcibly to the automobile 1 as a target for driving by the driver 2 in an inappropriate physical condition state for driving. For example, the management server device 40 can perform control so as to effect a temporary stop (driving can be performed again on the same day) in a case where a rest of the driver for a predetermined time restores the physical condition of the driver or effect a stop (driving cannot be performed again on the same day) in a case where it is determined that the rest will not restore the physical condition.

In addition, the management server device 40 accumulates the received writing trajectory information of the operation log, and also extracts the writing trajectory information of a specific pattern included in the operation log, detects a mental change in mood or emotion of the driver 2 who generates the operation log, and determines whether the driver 2 is in an inappropriate mental state for driving on the basis of the mental change. In the present embodiment, it is specified that the driver 2 write a signature of an own name in own handwriting in the operation log, and the signature is used as the specific pattern for grasping the mental change in the driver 2.

Therefore, in the present embodiment, writing trajectory information detected and obtained in the tablet terminal when each driver 2 signs the own name by using the electronic pen while in an appropriate mental state for driving the automobile, the appropriate mental state being a relaxed state, a concentrated state, or the like, is registered as comparative reference information (normal time information) in the management server device 40. The comparative reference information is stored as a specific pattern writing trajectory information database.

It is to be noted that an example of the specific pattern is not limited to the signature of the name of the driver as in the present embodiment, and that the specific pattern may be any writing pattern as long as the writing pattern is a predetermined writing pattern. In addition, all of the writing trajectory information of the operation log may of course be set as the specific pattern.

The management server device 40 analyzes the writing trajectory information of the signature as the specific pattern in the received writing trajectory information of the operation log of the driver 2 by using the data stored in the specific pattern writing trajectory information database. The management server device 40 thereby checks whether the mental state of the driver 2 is changed from a mental state appropriate for driving the automobile to an inappropriate mental state.

In this case, in the present embodiment, the management server device 40 is devised to be able to grasp not only whether the mental state of the driver 2 is changed but also what emotional state the changed mental state is by cooperating with the emotion server device 50. Specifically, as will be described later, the emotion server device 50 includes an emotion estimation database that stores emotional states such as a relaxed state, a concentrated state, an irritated state, and an angered state, and writing trajectory information at times of the respective emotional states in association with each other.

In the present embodiment, the management server device 40 sends an emotional state estimation request including the writing trajectory information of the signature as the specific pattern to the emotion server device 50. Receiving this emotional state estimation request, the emotion server device 50 returns, to the management server device 40, a result of estimation of an emotional state at a time of the received writing trajectory information of the signature by using the writing trajectory information of the signature included in the emotional state estimation request and information in the emotion estimation database. Incidentally, instead of sending only the writing trajectory information of the signature as the specific pattern from the management server device 40 to the emotion server device 50, a configuration may of course be adopted such that information as the writing trajectory information of the operation log is sent to the emotion server device 50, and the emotion server device 50 estimates the emotional state.

The management server device 40 determines whether the mental state of the driver 2 is changed from a mental state appropriate for the driving of the automobile 1 to an inappropriate mental state from a result of analysis using the writing trajectory information of the signature as the specific pattern and the data stored in the specific pattern writing trajectory information database and the estimated emotional state obtained from the emotion server device 50.

Then, the management server device 40 transmits an alarm to a driver 2 who is determined to be in an inappropriate state with regard to the driving of the automobile 1 in a result of determination of the mental state on the basis of the writing trajectory information of the signature as the specific pattern even when the driver 2 is determined not to be in an inappropriate state with regard to the driving of the automobile 1 in a result of determination of the physical condition on the basis of the biological information of the driver 2. The management server device 40 thereby prompts the driver 2 to suspend or stop the driving of the automobile 1. Further, together with this alarm transmission, the management server device 40 sends control information for performing remote control so as to temporary stop or stop traveling forcibly to the automobile 1 as a target for driving by the driver 2 in an inappropriate mental state for driving.

Description will next be made of a more detailed example of configuration of each constituent element of the operator state determining system configured as described above.

### <Example of Configurations of Tablet Terminal 10 and Electronic Pen 20>

In the present embodiment, the operation log is recorded and stored on predetermined paper (operation log paper) by writing input to the paper, and writing trajectory information as electronic data of a writing trajectory of the writing input can be obtained and retained by using functions of a position detecting circuit of the tablet terminal 10.

Therefore, in the present example, the tablet terminal 10 is configured to be able to lock the paper in a state in which the paper is placed on a position detecting sensor of the position detecting circuit. Then, the electronic pen 20 is configured to have a function capable of marking the paper as writing stationery, or a function of a ballpoint pen in the present example.

FIG. 2 and FIGS. 3A to 3D depict an example of a hardware configuration of the tablet terminal 10 in the present first embodiment together with an example of a configuration of the electronic pen 20. FIG. 2 is a diagram of assistance in explaining an external appearance of the tablet terminal 10 and the electronic pen 20 in the present example. FIGS. 3A to 3D are exploded configuration views of assistance in explaining an example of a hardware configuration of the tablet terminal 10.

As depicted in FIG. 2, the tablet terminal 10 in the present embodiment uses a surface for indication input by the electronic pen 20 (detection region of the position detecting sensor) as a paper mounting surface 10s. Then, the tablet terminal 10 in the present example has a clip member 11 on an upper end portion of the mounting surface 10s, the clip member 11 fixing operation log paper 6 on the mounting surface 10s as the indication input surface of the tablet terminal 10 by sandwiching the operation log paper 6, and the tablet terminal 10 has an external appearance similar to that of stationery referred to as a binder or the like and used widely.

In the present embodiment, the position detecting device 100 of an electromagnetic induction type is included on the inside of the tablet terminal 10 which inside is on the undersurface side of the mounting surface 10s. As depicted in FIGS. 3A to 3D, the position detecting device 100 includes a position detecting sensor 110, a coordinate data generating circuit 120, and a controller circuit 130.

As depicted in FIGS. 3A to 3D, the tablet terminal 10 includes an upper cover (upper plate) 12 (FIG. 3A), the position detecting sensor 110 (FIG. 3B) for interacting with the electronic pen 20 by electromagnetic induction coupling, a shield sheet 13 (FIG. 3C), and a sensor cover 14 (FIG. 3D) in order from above with the mounting surface 10s side as an upper side, the mounting surface 10s serving as an indication input surface. Then, as depicted in FIG. 3C, the coordinate data generating circuit 120 and the controller circuit 130 connected to the position detecting sensor 110 with the shield sheet 13 interposed therebetween are provided to the lower side of the shield sheet 13.

The coordinate data generating circuit 120 detects the coordinate data of a position indicated by the electronic pen 20 and receives and obtains pen pressure data and a pen identification (ID) on the basis of output from the position detecting sensor 110.

The controller circuit 130 is connected to the coordinate data generating circuit 120. The controller circuit 130 has a function of transmitting the pen pressure data and the pen ID to the management server device 40 of the determining device 4 through the communication network 5 together with the coordinate data detected by the coordinate data generating circuit 120.

In addition, the controller circuit 130 includes a short-range wireless communication circuit for wirelessly communicating with the smart watch 30, and has functions of receiving the biological information sent from the smart watch 30 and transmitting the biological information to the management server device 40 of the determining device 4 through the communication network 5 together with the coordinate data position indicated by the electronic pen 20 and the pen pressure data and the pen ID. Then, the wireless communication circuit of the controller circuit 130 is provided with a transmission antenna AT.

The upper cover 12 is formed of a material that transmits a signal (radio wave) transmitted from the electronic pen 20, the material being, for example, a synthetic resin, a glass, a ceramic, or the like. A surface side of the upper cover 12 which surface side is exposed to the outside is the mounting surface 10s that also serves as the indication input surface of the position detecting device 100.

The sensor cover 14 is formed of a synthetic resin, a metal, or the like, and is configured in a thin box shape whose upper surface forms an opening portion. The position detecting sensor 110, the coordinate data generating circuit 120, the controller circuit 130, and the shield sheet 13 are housed within the sensor cover 14.

In the present example, a wall portion of the sensor cover 14 is configured to project on the periphery of the mounting surface 10s, and thereby act as a position regulating member for the operation log paper 6. That is, when the paper is mounted on the mounting surface 10s, the wall portion of the sensor cover 14 regulates the mounting position of the operation log paper 6.

The clip member 11 is formed by a constituent part as a plate-shaped body and a spring member and is configured to sandwich the operation log paper 6 by elastically pressing the operation log paper 6 against the mounting surface 10s. In the present embodiment, the operation log paper 6 is printed with a paper form on which specified items are to be entered in predetermined sections.

Hence, in a state in which the operation log paper 6 is locked on the mounting surface 10s so as to be pressed and sandwiched by the clip member 11 while the operation log paper 6 is mounted on the mounting surface 10s so as to be regulated in position, the operation log paper 6 coincides with an indication input region of the position detecting sensor 110 of the position detecting device 100 of the tablet terminal 10. Then, each section of the paper form printed on the operation log paper 6 is included in the respective determined coordinate range position of the position detecting sensor 110. Hence, writing input of the signature to a signature space 6a for the driver which signature space is formed on the paper form printed on the operation log paper 6 can be recognized in a distinguished manner as coordinate data within the signature space 6a from coordinate data output information detected in the position detecting device 100.

The tablet terminal 10 in the present embodiment includes a rechargeable battery, which is not depicted in the figures. When a power button 15 is turned on, a driving voltage is supplied from the battery. In addition, as depicted in FIG. 2 and FIGS. 3A to 3D, the tablet terminal 10 is provided with an end button 16 that is depressed when the driver completes writing input to the operation log by using the electronic pen 20. The depression of the end button 16 is a trigger for transmitting the writing trajectory information of the operation log to which the writing input has been performed to the management server device 40.

Incidentally, the clip member 11 of the tablet terminal 10 in the present embodiment is provided with indicators 11a to 11c and a speaker 11sp. In the present embodiment, as depicted in FIG. 2 and FIGS. 3A to 3D, the end button 16, the indicators 11a to 11c, and the speaker 11sp are provided in the constituent part as the plate-shaped body of the clip member 11. The indicators 11a to 11c in the present example are formed by a light emitting diode (LED).

The indicator 11a is for notifying a turned-on state of power. The indicator 11b is for notifying a state of wireless connection between the tablet terminal 10 and the management server device 40. The indicator 11c is for checking whether the operation log paper 6 mounted on the mounting surface 10s is mounted on the mounting surface 10s correctly. The speaker 11sp is for notifying various states to a user by alarm sound. The speaker 11sp may be used in combination with notifications by the above-described indicators 11a to 11 c.

In addition, a back side of the plate-shaped body of the clip member 11 of the tablet terminal 10 is provided with a paper mounting sensor 11s that detects whether or not the operation log paper 6 is sandwiched and locked by the clip member 11.

As depicted in FIGS. 3A to 3D, the power button 15, the end button 16, the indicators 11a to 11c, the speaker 11sp, and the paper mounting sensor 11s are connected to the controller circuit 130 through a connection connector CN.

As depicted in FIG. 2, the electronic pen 20 in the present embodiment has a ballpoint pen function in the present example as a function of being able to leave visually recognizable handwriting on the paper 6 by including a core body 21 filled with an ink for writing and a tip portion (pen tip) 22 provided to an end portion of the core body 21.

The electronic pen 20 in the present embodiment transmits and receives signals to and from the position detecting sensor 110 of the tablet terminal 10 by an electromagnetic induction system, and thereby makes the position detecting device 100 of the tablet terminal 10 detect a position indicated by the electronic pen 20. A configuration of an electronic pen 20 of a publicly known electromagnetic induction type can be used as a mechanical configuration of the electronic pen 20 in the present embodiment except that the core body 21 has a ballpoint pen function. Thus, illustration of the mechanical configuration will be omitted herein.

In addition, the electronic pen 20 in the present embodiment has a pen pressure detector that detects a pen pressure applied to the pen tip, and has means for transmitting information regarding the detected pen pressure to the tablet terminal 10. Though not depicted, the pen pressure detector in the present example has a configuration using a semiconductor element that varies in capacitance according to the pen pressure as disclosed in Japanese Patent Laid-Open No. 2013-161307, for example. Incidentally, the pen pressure detector can also have a configuration of a variable capacitance capacitor that changes in capacitance according to the pen pressure, the configuration using pen pressure detector of a well-known structural configuration described in Patent Document, Japanese Patent Laid-Open No. 2011-186803, for example.

In addition, in the present embodiment, the electronic pen 20 has a pen ID memory that stores unique identification information (referred to as a pen ID), and has means for transmitting the pen ID to the tablet terminal 10.

Then, in the present example, information regarding the pen pressure and the pen ID from the electronic pen 20 is transmitted as an amplitude-shift keying (ASK) modulated signal, as will be described later, to the tablet terminal 10 through interaction by electromagnetic induction coupling. Incidentally, the information regarding the pen pressure and the pen ID from the electronic pen 20 can also be transmitted wirelessly by using short-range wireless communication means of the Bluetooth (registered trademark) standard, for example, rather than interaction by electromagnetic induction coupling.

In the present embodiment, the drivers 2₁, . . . , 2ₙ respectively use the respective electronic pens 20₁, . . . , 20ₙ for the drivers themselves. The respective pen IDs of the electronic pens 20₁, . . . , 20ₙ therefore play a role as respective pieces of identification information of the drivers 2₁, . . . , 2ₙ.

### <Example of Electronic Circuit Configurations of Position Detecting Device 100 of Tablet Terminal 10 and Electronic Pen 20>

FIG. 4 is a diagram depicting an equivalent circuit of an electronic circuit possessed by the electronic pen 20 and an example of a circuit configuration of the position detecting device 100 of the tablet terminal 10 that performs position detection and pen pressure detection by an electromagnetic induction system in conjunction with the electronic pen 20.

The position detecting device 100 of the tablet terminal 10 in the example of FIG. 4 includes the position detecting sensor 110, the coordinate data generating circuit 120, and the controller circuit 130. In the position detecting sensor 110, an X-axis direction loop coil group 111 and a Y-axis direction loop coil group 112 are formed in a stacked manner, and a selecting circuit 113 is provided which sequentially selects one loop coil in the two loop coil groups 111 and 112.

The electronic pen 20 includes a signal control circuit formed by an IC 200 (e.g., including a processor and a memory storing instructions that, when executed by the processor, cause the IC 200 to perform the functions of the IC 200 described herein) and is configured to obtain a driving voltage for driving the IC 200 from an excitation signal transmitted from an excitation coil 114 provided to the position detecting sensor 110 of the position detecting device 100 of the tablet terminal 10. Incidentally, while description will be made supposing that in FIG. 4, as an example, the loop coil groups 111 and 112 of the position detecting sensor 110 are used only to receive an electromagnetic coupling signal from the electronic pen 20, this does not exclude the driving of the signal control circuit provided to the electronic pen 20 in place of the excitation coil 114 by performing electromagnetic coupling with the electronic pen 20. In addition, this does not exclude transmission of a signal such as predetermined control data to the signal control circuit provided to the electronic pen 20.

The position detecting sensor 110 of the position detecting device 100 of the tablet terminal 10 in the example of FIG. 4 has the excitation coil 114 disposed so as to surround the X-axis direction loop coil group 111 and the Y-axis direction loop coil group 112. As depicted in FIG. 4, the excitation coil 114 is connected to a drive circuit 122, and the drive circuit 122 is connected to an oscillating circuit 121 that oscillates at a frequency fo.

The drive circuit 122 is controlled by a processing control circuit 128 constituted by a microcomputer (e.g., including a processor and a memory storing instructions that, when executed by the processor, cause the processing control circuit 128 to perform the functions of the processing control circuit 128 described herein). The processing control circuit 128 controls the supply of an oscillating signal of the frequency fo from the oscillating circuit 121 to the excitation coil 114 by controlling the drive circuit 122, and thereby controls signal transmission from the excitation coil 114 to the electronic pen 20.

The selecting circuit 113 is selectively controlled by the processing control circuit 128 to select one loop coil. An induced voltage generated in the loop coil selected by the selecting circuit 113 is amplified by a receiving amplifier 123, and is then supplied to a band-pass filter 124, which extracts only a component of the frequency fo. The band-pass filter 124 supplies the extracted component to a detecting circuit 125.

The detecting circuit 125 detects the component of the frequency fo and supplies a direct-current signal corresponding to the detected component of the frequency fo to a sample and hold circuit 126. The sample and hold circuit 126 retains a voltage value in predetermined timing of the output signal of the detecting circuit 125, or specifically in predetermined timing in a reception period, and sends out the voltage value to an AD converter circuit 127. The AD converter circuit 127 converts the analog output of the sample and hold circuit 126 into a digital signal, and outputs the digital signal to the processing control circuit 128. The processing control circuit 128 supplies the signal in the predetermined timing to the sample and hold circuit 126.

Then, the processing control circuit 128 determines whether or not the loop coil selected by the selecting circuit 113 is a loop coil at a position indicated by the electronic pen 20 by determining whether or not the digital signal from the AD converter circuit 127 is a value exceeding a predetermined threshold value. The processing control circuit 128 detects the position indicated by the electronic pen 20 on the basis of the determination. Incidentally, the processing control circuit 128 has a clock function, and information regarding the detected position indicated by the electronic pen includes time information regarding a time point of the detection. Hence, information regarding a writing trajectory formed by a sequence of information regarding the position indicated by the electronic pen also includes information regarding temporal changes, so that the formation speed of the writing trajectory can be detected.

In addition, as will be described later, separately from the detection of the position indicated by the electronic pen 20, the processing control circuit 128 detects interruptions of the signal from the electronic pen 20 as a digital signal of several bits, and thereby detects the pen pressure and detects the pen ID.

In addition, in the present example, though not depicted, the position detecting device 100 of the tablet terminal 10 has a function of detecting the inclination of the electronic pen 20 and the height position of the electronic pen 20. Here, the inclination of the electronic pen 20 is the inclination of the electronic pen 20 with respect to the input surface of the position detecting sensor 110. In addition, the height position of the electronic pen 20 is a height position from the input surface of the position detecting sensor 110. The inclination and the height position of the electronic pen 20 can be detected by using a publicly known method. Thus, description of the detecting method will be omitted here.

The tablet terminal 10 associates, with the pen ID, writing trajectory information including information obtained by adding time information to the information regarding the detected position indicated by the electronic pen 20, information regarding the pen pressure, information regarding the inclination, and information regarding the height position as described above. The tablet terminal 10 supplies the resulting writing trajectory information to the controller circuit 130.

The controller circuit 130 temporarily stores the received writing trajectory information in a transmission information generating circuit 131 (e.g., including a processor and a memory storing instructions that, when executed by the processor, cause the transmission information generating circuit 131 to perform the functions of the transmission information generating circuit 131 described herein). In addition, the controller circuit 130 receives biological information such as the heartbeat, and body temperature of the driver from the smart watch 30 by a short-range wireless communication circuit 132, supplies the biological information to the transmission information generating circuit 131, and temporarily stores the biological information therein.

As depicted in FIG. 4, the smart watch 30 is provided with a short-range wireless communication circuit 30T that communicates with the short-range wireless communication circuit 132 of the controller circuit 130. The transmission information generating circuit 131 of the controller circuit 130 generates transmission information as information obtained by associating the pen ID with the writing trajectory information and the biological information.

Then, the controller circuit 130 transmits the information obtained by associating the pen ID with the writing trajectory information and the biological information, the information being temporarily stored in the transmission information generating circuit 131, to the management server device 40 through a wireless communication circuit 133. A circuit configuration of the electronic pen 20 is depicted enclosed by a dotted line in FIG. 4. However, in the example of FIG. 4, a circuit part for detection of the inclination of the electronic pen 20 in the position detecting device 100 is omitted.

Specifically, in the electronic pen 20 in the example of FIG. 4, a resonance circuit 203 is formed by connecting a capacitor 202 in parallel with a coil 201. Then, a switch circuit 204 is connected in parallel with the resonance circuit 203. The switch circuit 204 is configured to be subjected to on-off control by the IC 200. When the switch circuit 204 is off, a resonant operation of the resonance circuit 203 with a signal from the position detecting sensor 110 (alternating-current signal from the excitation coil 114) is performed. However, when the switch circuit 204 is on, the capacitor 202 connected in parallel with the coil 201 is short-circuited, and thus the resonant operation of the resonance circuit 203 with the signal from the position detecting sensor 110 is off.

Then, the IC 200 is configured to operate on a power supply voltage Vcc obtained by rectifying the alternating-current signal in a rectifier circuit (power supply circuit) 207 including a diode 205 and a capacitor 206, the alternating-current signal being received in the resonance circuit 203 from the position detecting sensor 110 of the position detecting device 100 of the tablet terminal 10 by electromagnetic induction. The IC 200 is connected to the resonance circuit 203 via a capacitor 208 to monitor operation conditions of the resonance circuit 203. By monitoring the operation conditions of the resonance circuit 203, the IC 200 can detect conditions of electromagnetic coupling with the excitation coil 114 of the position detecting sensor 110 or a signal such as control data transmitted from the position detecting sensor 110 of the position detecting device 100 by using the two loop coil groups 111 and 112, though description thereof will be omitted in the present example, and accordingly perform desired operation control.

As depicted in FIG. 4, the IC 200 is connected with a variable capacitance capacitor Cv formed by a pen pressure detector. The IC 200 detects the pen pressure applied to a tip end 20T of the electronic pen 20 from a value of capacitance of the variable capacitance capacitor Cv. Then, the IC 200 converts the detected pen pressure into a digital signal of a plurality of bits and controls the switch circuit 204 according to the digital signal corresponding to the pen pressure. The IC 200 thereby transmits pen pressure information as additional information of a position detection signal to the position detecting device 100 of the tablet terminal 10.

In addition, the IC 200 is connected with an ID memory 210 that stores the pen ID as the identification information of the electronic pen 20. The IC 200 transmits also the pen ID as additional information of the position detection signal to the position detecting device 100 of the tablet terminal 10 together with the pen pressure information by controlling the switch circuit 204 according to the digital signal of the pen ID stored in the ID memory 210.

Description will be made of a position detecting operation and an operation of detecting the pen pressure information and the pen ID in the electronic pen 20 and the position detecting device 100 of the tablet terminal 10 configured as described above.

The processing control circuit 128 of the coordinate data generating circuit 120 of the position detecting device 100 first transmits a signal from the excitation coil 114 to the electronic pen 20 for a predetermined time by driving the drive circuit 122. Thereafter, the processing control circuit 128 sends out a signal in a burst state from the excitation coil 114 by driving the drive circuit 122, and performs, for all of loop coils of the X-axis direction loop coil group 111, processing of making the selecting circuit 113 sequentially select one loop coil of the X-axis direction loop coil group 111. Meanwhile, the electronic pen 20 receives the signal in a burst state by the resonance circuit 203 and feeds back the signal to the position detecting sensor 110 of the position detecting device 100 of the tablet terminal 10. Thus, the processing control circuit 128 obtains the X-coordinate value of a position indicated by the electronic pen 20 by detecting the fed-back signal in a burst state as a position detection signal.

Next, the processing control circuit 128 transmits a signal from the excitation coil 114 to the electronic pen 20 for a predetermined time again by driving the drive circuit 122. Thereafter, the processing control circuit 128 sends out a signal in a burst state from the excitation coil 114 by driving the drive circuit 122, and performs, for all of loop coils of the Y-axis direction loop coil group 112, processing of making the selecting circuit 113 sequentially select one loop coil of the Y-axis direction loop coil group 112. Then, the processing control circuit 128 obtains the Y-coordinate value of the position indicated by the electronic pen 20 by detecting the signal in a burst state fed back from the electronic pen 20 as a position detection signal.

After detecting the position indicated by the electronic pen 20 as described above, in order to detect the pen pressure information and the pen ID as additional information from the electronic pen 20, the processing control circuit 128 performs transmission continued for a predetermined time or more from the excitation coil 114, and thereafter continually performs transmission and reception of a signal in a burst state the number of times corresponding to the number of bits of the digital signal of the additional information from the electronic pen 20. At this time, the selecting circuit 113 receives the signal by selecting a loop coil closest to the electronic pen 20 (which loop coil may be either of an X-axis direction loop coil or a Y-axis direction loop coil) according to the detected coordinate value.

On the other hand, the IC 200 of the electronic pen 20 performs on-off control of the switch circuit 204 in synchronism with the transmission and reception of the signal from the position detecting device circuit 100 of the tablet terminal 10 according to the digital signal of the additional information including the pen pressure information, which is obtained in correspondence with the capacitance of the variable capacitance capacitor Cv constituting the pen pressure detector, and the pen ID. When the switch circuit 204 is off, the resonance circuit 203 can return the signal transmitted from the position detecting device 100 to the position detecting device 100. Thus, the loop coil of the position detecting device 100 receives this signal. On the other hand, when the switch circuit 204 is on, the resonant operation of the resonance circuit 203 is in a prohibited state. Therefore, the signal is not returned from the resonance circuit 203 to the position detecting device 100, and the loop coil of the position detecting device 100 does not receive the signal.

The processing control circuit 128 of the coordinate data generating circuit 120 of the position detecting device 100 can receive the digital signal of a plurality of bits corresponding to the pen pressure information and the pen ID and thus detect the pen pressure information and the pen ID from the electronic pen 20 by determining the presence or absence of the received signal the number of times which number is equal to the number of bits of the digital signal of the additional information. Hence, the electronic pen 20 transmits the pen pressure information and the pen ID as a signal modulated by amplitude shift keying (ASK) to the position detecting device 100 of the tablet terminal 10.

The processing control circuit 128 of the position detecting device 100 detects the information regarding the position indicated by the electronic pen 20 and detects the pen pressure information and the pen ID from the electronic pen 20, and supplies the detected position indicated by the electronic pen 20 as well as the detected pen pressure information and the detected pen ID to the controller circuit 130.

### <Example of Hardware Configuration of Management Server Device 40>

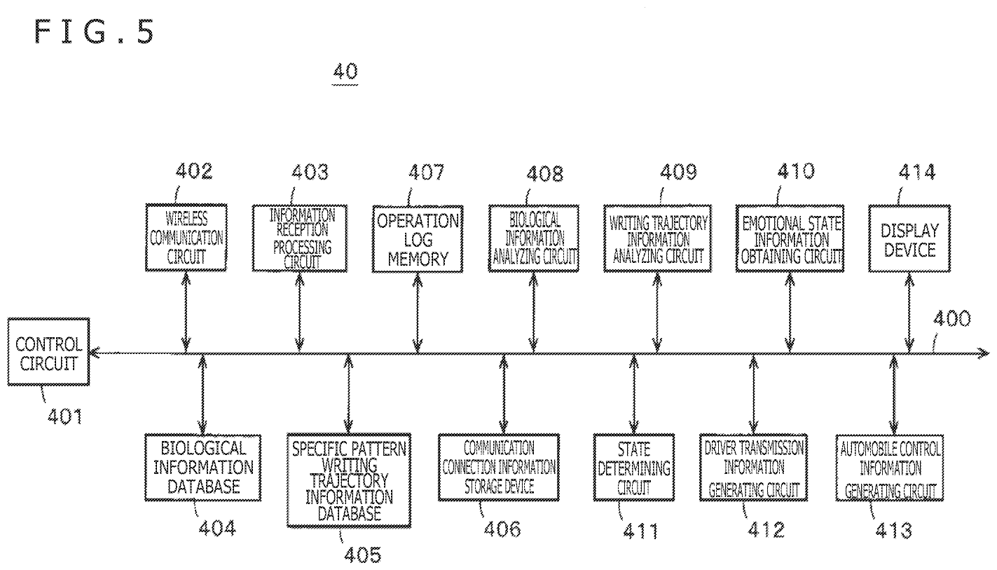

FIG. 5 is a block diagram depicting an example of a configuration of the management server device 40 of the operator state determining system according to the present embodiment.

As depicted in FIG. 5, the management server device 40 in the present embodiment is configured by connecting, to a control circuit 401 constituted by a computer (e.g., including a processor and a memory storing instructions that, when executed by the processor, cause the control circuit 401 to perform the functions of the control circuit 401 described herein) through a system bus 400, each of a wireless communication circuit 402, an information reception processing circuit 403, a biological information database 404, a specific pattern writing trajectory information database 405, a communication connection information storage device 406, an operation log memory 407, a biological information analyzing circuit 408, a writing trajectory information analyzing circuit 409, an emotional state information obtaining circuit 410, a state determining circuit 411, a driver transmission information generating circuit 412, an automobile control information generating circuit 413, and a display device 414.

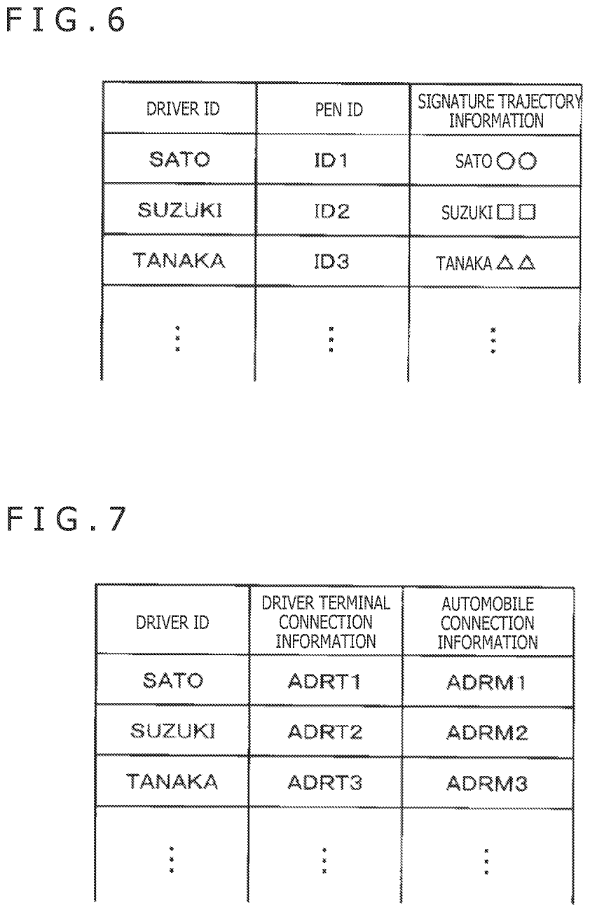

As depicted in FIG. 6, the specific pattern writing trajectory information database 405 stores the respective driver IDs (in the present example, the driver names) of the drivers 2₁, . . . , 2ₙ, the respective pen IDs of the electronic pens 20₁, . . . , 20ₙ, and signature writing information as writing trajectory information when the drivers 2₁, . . . , 2ₙ respectively write own names as signatures by the respective electronic pens 20₁, . . . , 20ₙ in association with one another.

In this case, the signature writing information is obtained by writing input performed when each of the drivers 2₁, . . . , 2ₙ is in an appropriate state for driving an automobile, the appropriate state being a concentrated state, a relaxed state, or the like.

Coordinate data constituting the writing trajectory information accompanies time information. The writing speed of each character of the signature writing information can be detected from the time information.

Incidentally, because the driver IDs and the pen IDs are stored in association with each other in the specific pattern writing trajectory information database 405, reference to the specific pattern writing trajectory information database 405 on the basis of a pen ID can obtain an associated (linked) driver ID.

Though not depicted, the biological information database 404 stores numerical value information regarding normal state ranges of the heartbeat, the body temperature, perspiration, and the like in the present example as the biological information in the respective normal states of the drivers 2₁, . . . , 2ₙ. In the present example, the biological information in the normal states is stored in association with the driver IDs linked to the respective pen IDs of the electronic pens 20₁, . . . , 20ₙ. Incidentally, while the biological information database 404 stores the biological information in the normal state of each of the drivers 2₁, . . . , 2, in the present example, information regarding a normal state which information is generated from the biological information of a large number of unspecified subjects rather than each driver 2 may be stored and used.

As depicted in FIG. 7, the communication connection information storage device 406 stores connection information for connecting to each of the tablet terminals 10₁ to 10ₙ through the wireless communication circuit 402 and connection information for connecting to each of the automobiles 1₁ to 1ₙ through the wireless communication circuit 402 in association with each other for each of the driver IDs.

The wireless communication circuit 402 is for wireless communication with each of the tablet terminals 10₁ to 10ₙ through the communication network 5.

The information reception processing circuit 403 distinguishes and recognizes, from information received from each of the tablet terminals 10₁ to 10ₙ, the biological information, the writing trajectory information, signature information as the specific pattern writing trajectory information in the writing trajectory information, the time information, and the pen ID, and distributes each piece of information to each circuit that needs the information.

In this case, the information reception processing circuit 403 obtains the driver ID by referring to the specific pattern writing trajectory information database 405 on the basis of the pen ID, and adds also the obtained driver ID to the information to be distributed.

The operation log memory 407 stores all of the received writing trajectory information of an operation log in association with the driver ID and/or the pen ID and accompanying date and time information for management in the taxi company.

The biological information analyzing circuit 408 checks whether an abnormality has occurred in the physical condition of the driver 2 who has transmitted the writing trajectory information of the operation log (hereinafter written as the driver 2S) on the basis of whether the value of the heartbeat, the body temperature, perspiration, or the like as the biological information of the driver 2S is within a numerical range of a normal state of the heartbeat, the body temperature, perspiration, or the like of the corresponding driver in the biological information database 404. Then, the biological information analyzing circuit 408 sends a result of the analysis to the state determining circuit 411.

The writing trajectory information analyzing circuit 409 in the present embodiment extracts writing trajectory information in the signature space from the received writing trajectory information of the operation log, compares the writing trajectory information in the signature space by reference to the written signature information of the driver 2S in the specific pattern writing trajectory information database 405, and checks whether the mental state of the driver 2S is changed to an inappropriate state for driving an automobile. Then, the writing trajectory information analyzing circuit 409 sends a result of the analysis to the state determining circuit 411. Incidentally, as also described above, the writing trajectory information analyzing circuit 409 may analyze all of the received writing trajectory information of the operation log, and check whether the mental state of the driver 2S is changed to an inappropriate state for driving an automobile.

The emotional state information obtaining circuit 410 in the present embodiment extracts the writing trajectory information in the signature space from the received writing trajectory information of the operation log, includes the extracted writing trajectory information in the signature space in an emotional state estimation request together with the pen ID or the driver ID, sends the emotional state estimation request to the emotion server device 50 through the communication network 5, and waits for a reply thereto.

When the emotion server device 50 receives the emotional state estimation request, the emotion server device 50 estimates the emotional state of the driver 2S from the writing trajectory information of the signature which writing trajectory information is included in the emotional state estimation request, as will be described later. The emotion server device 50 then sends a reply to the management server device 40. The emotional state information obtaining circuit 410 sends the state determining circuit 411 information regarding the emotional state of the driver 2S, the information being obtained from the emotion server device 50.

The state determining circuit 411 determines whether the physical condition state and/or the mental state of the driver 2S is changed to an inappropriate state for driving an automobile from the analysis result from the biological information analyzing circuit 408, the analysis result from the writing trajectory information analyzing circuit 409, and the information regarding the emotional state from the emotional state information obtaining circuit 410. Then, the state determining circuit 411 sends a result of the determination to the driver transmission information generating circuit 412 and the automobile control information generating circuit 413. In addition, the state determining circuit 411 sends the determination result as display information to the display device 414.

The driver transmission information generating circuit 412 generates driver transmission information corresponding to the determination result from the state determining circuit 411. In this case, when the determination result from the state determining circuit 411 indicates that the driver is in an inappropriate state for driving an automobile, the driver transmission information generating circuit 412 generates driver transmission information including an alarm for not permitting the continuation of driving, for example, an alarm voice message such as “Your physical condition or mental state is not appropriate for driving. Please suspend driving for a while,” “Your physical condition or mental state is not appropriate for driving. Please stop driving,” or the like.

Then, the driver transmission information generating circuit 412 transmits information regarding the generated alarm voice message to the tablet terminal 10 of the driver 2S through the wireless communication circuit 402 and through the communication network 5. The driver transmission information generating circuit 412 thereafter sends a communication ending request to the tablet terminal 10, and performs processing for ending communication. In this case, when the communication with the tablet terminal 10 of the driver 2S is already disconnected at a time of sending the alarm, the driver transmission information generating circuit 412 forms a communication path by using connection information of the tablet terminal 10 of the driver 2S which connection information is stored in the communication connection information storage device 406, and sends the driver transmission information.

Then, when the determination result from the state determining circuit 411 indicates that the driver 2S is “not in an inappropriate state for driving an automobile,” the driver transmission information generating circuit 412 directly performs the processing for ending communication with the tablet terminal 10. Incidentally, when the determination result from the state determining circuit 411 indicates that the driver 2S is “not in an inappropriate state for driving an automobile,” the driver transmission information generating circuit 412 may send driver transmission information for permitting the continuation of driving, for example, a voice message such as “Thank you. Please continue work while continuing paying attention to safe driving” or the like.

When the determination result from the state determining circuit 411 indicates that the driver 2S is “in an inappropriate state for driving an automobile,” the automobile control information generating circuit 413 generates a control signal to make it impossible for the automobile 1 to travel, transmits the generated control signal to the automobile 1 as a target for driving by the driver 2S through the wireless communication circuit 402 and through the communication network 5. At this time, the automobile control information generating circuit 413 obtains the connection information of the automobile 1 as a target for driving by the driver 2S which connection information is stored in the communication connection information storage device 406, forms a communication path by using the connection information, and sends the control signal to the automobile.

Incidentally, each automobile 1 has a functional circuit that performs control so as to set the automobile 1 in a driving stop state by, for example, interrupting the supply of power from a battery to parts related to traveling, for example, when receiving the control signal from the management server device 40.

The display device 414 includes a display screen. The display device 414 receives the writing trajectory information of the operation log from the information reception processing circuit 403, and displays the writing trajectory information of the operation log on the display screen. The management server device 40 in the present example is disposed in the taxi company. A person in charge of driver state management who manages the physical condition states and the mental states of the drivers of the taxi company views display information of the writing trajectory of the operation log which display information is displayed on the display screen. By viewing the writing trajectory of the operation log on the display screen of the display device 414 and visually grasping whether the writing trajectory of the operation log is different from that at a normal time, the person in charge of driver state management can infer an abnormality in the physical condition state or the mental state of the driver who has generated the operation log.

Then, because the display information of the determination result from the state determining circuit 411 is sent to the display device 414 in the present embodiment, the display screen displays the state of the driver in addition to the display information of the writing trajectory of the operation log. Hence, the person in charge of driver state management at the taxi company can grasp the state of the driver on the display screen of the display device 414. As required, the person in charge of driver state management can directly call the driver by using a mobile telephone terminal or the like, and inquire about the physical condition state and the mental state or give advice.

Incidentally, because the above-described management server device 40 stores the writing trajectory information of the signature by the driver 2, the management server device 40 can also perform authentication of the driver 2S by comparing the writing trajectory information of the signature in the signature space of the operation log sent from the tablet terminal and the stored writing trajectory information of the signature of the driver 2 with each other.

Incidentally, the control circuit 401 can implement, as software processing, a program that performs functions of each of the information reception processing circuit 403, the biological information analyzing circuit 408, the writing trajectory information analyzing circuit 409, the emotional state information obtaining circuit 410, the state determining circuit 411, the driver transmission information generating circuit 412, and the automobile control information generating circuit 413 depicted in FIG. 5. However, it is needless to say that instead of converting all of the above-described circuits into the processing functions of the program, a part of the above-described circuits may be configured as a constituent circuit of hardware.

### [Example of Configuration of Emotion Server Device 50]

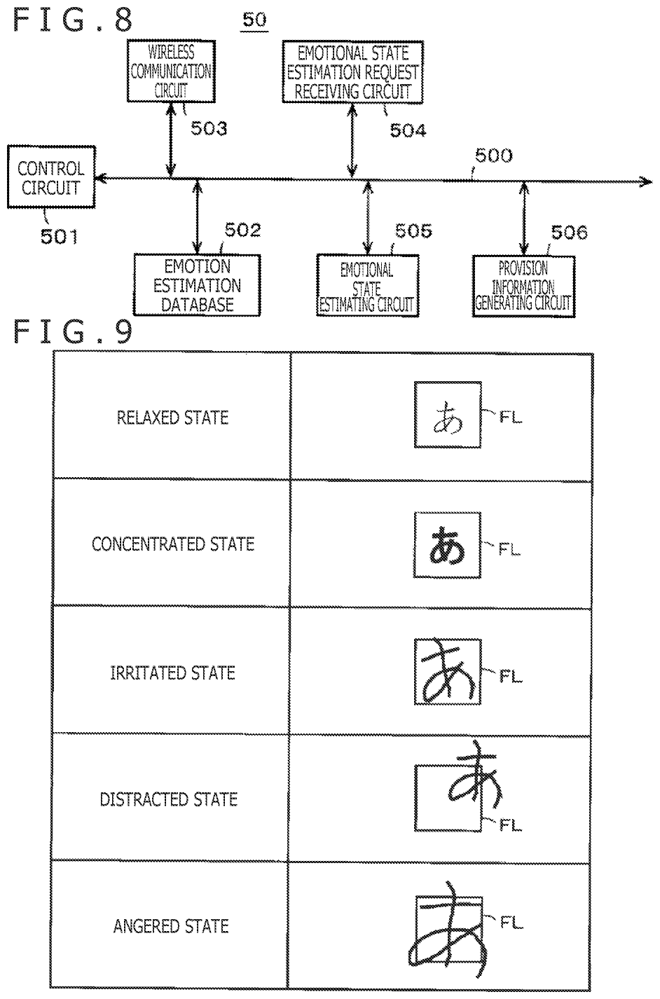

FIG. 8 is a block diagram depicting an example of a hardware configuration of the emotion server device 50. The emotion server device 50 in this example of FIG. 8 is configured by connecting an emotion estimation database 502, a wireless communication circuit 503, an emotional state estimation request receiving circuit 504, an emotional state estimating circuit 505, and a provision information generating circuit 506 to a control circuit 501 constituted by a computer (e.g., including a processor and a memory storing instructions that, when executed by the processor, cause the control circuit 501 to perform the functions of the control circuit 501 described herein) through a system bus 500.

The emotion estimation database 502 stores various emotional states and pen state information of the electronic pen in the respective emotional states in association with each other for each of the drivers 2₁, . . . , 2ₙ. Necessary information for the data stored in the emotion estimation database 502 is collected from each of the drivers 2₁, . . . , 2ₙ in advance, and the data stored in the emotion estimation database 502 is constructed on the basis of the collected information.

Specifically, for example, the drivers 2₁, . . . , 2ₙ are respectively made to wear electroencephalographs, and are made to perform a writing input by using the respective tablet terminals 10₁ to 10ₙ and the respective electronic pens 20₁, . . . , 20ₙ.

Then, condition stimuli such as give rise to emotional states such, for example, as a “relaxed state,” a “concentrated state,” an “irritated state,” a “distracted state,” and an “angered state,” for example, applied to each of the drivers 2₁, . . . , 2ₙ, and each of the emotional states is made to continue for a time width of a predetermined time or more. At this time, it is confirmed by measurement using the electroencephalographs that each of the drivers 2₁, . . . , 2ₙ is in each of the emotional states.

Then, the drivers 2₁, . . . , 2ₙ are made to perform a writing input on the respective tablet terminals 10₁ to 10ₙ by using the electronic pens 20₁, . . . , 20ₙ in a predetermined time in which the drivers 2₁, . . . , 2ₙ are in the emotional state of each of the “relaxed state,” the “concentrated state,” the “irritated state,” the “distracted state,” and the “angered state.” Each of the tablet terminals 10₁ to 10ₙ is made to detect writing trajectory information (information regarding an indicated position and pen state information such as the inclination, and the height position of the electronic pen) at the times of the above-described emotional states. Then, the writing trajectory information in each of the emotional states which writing trajectory information is obtained by each of the tablet terminals 10₁ to 10ₙ is associated with the pen ID, and is sent to an emotion estimation database constructing device.

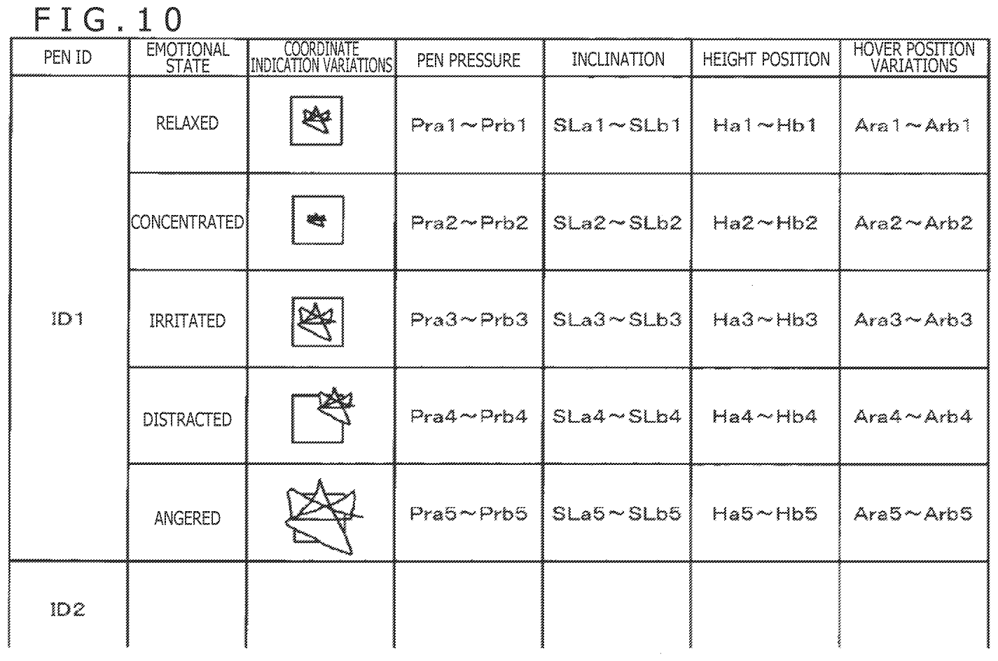

The emotion estimation database constructing device retains the writing trajectory information of each of the drivers 2₁, . . . , 2ₙ in such a manner as to be classified by the emotional states, which are respectively the “relaxed state,” the “concentrated state,” the “irritated state,” the “distracted state,” and the “angered state.” Then, the emotion estimation database constructing device calculates, as an existence range that is a characteristic value of each emotional state, a possible range of each of the information regarding the indicated position and the pen state information such as the inclination, and the height position of the electronic pen in the writing trajectory information in each of the emotional states. That is, an information value of each of pen states (change in movement of the indicated position, the pen pressure value, the inclination of the electronic pen, the height position, and the like) of the electronic pen during the writing input of the driver in each of the emotional states has a different possible range, that is, a different existence range according to each of the emotional states. Thus, the existence range of each piece of pen state information for each of the emotional states is calculated. The calculated existence range of each piece of pen state information for each of the emotional states is set as stored contents in the emotion estimation database depicted in FIG. 10 to be described later.

For example, the pen pressure applied to the electronic pen 20 at the time of the “relaxed state” has a relatively low average value, and has an existence range such that deviation from the average value is a relatively small value. In addition, the pen pressure applied to the electronic pen 20 at the time of the “irritated state” has a relatively high average value, and has an existence range such that deviation from the average value is a relatively large value.

Incidentally, the range information of pen state information is calculated by determining a range in which most of, for example, 90 percent of values of the pen state information at a time of a certain emotional state are included, that is, exist. The range information of the pen state information (information regarding the existence range) can be set as range information including the average value and the deviation described above.

In addition, in the present embodiment, the emotion estimation database constructing device stores also a “characteristic pattern at a time of character writing” by the electronic pen 20 in each emotional state of the driver 2. Therefore, in the present example, the driver 2 is made to write a predetermined character within a predetermined rectangular region at the time of each of the emotional states.

Specifically, as depicted in FIG. 9, for the driver 2, a character input quadrangular frame FL is displayed on the display screen of the tablet terminal 10, and the driver 2 is prompted to input a character within the input quadrangular frame FL. As depicted in FIG. 9, the driver 2 performs a writing input of a character requested to be input, in the present example “ [symbol] ” within the input quadrangular frame FL by the electronic pen 20 in each of the emotional states.

As depicted in FIG. 9, in the relaxed state or the concentrated state, the driver 2 performs a writing input so as to neatly include the requested character within the input quadrangular frame FL. That is, the central position of the character is in the vicinity of the central position of the input quadrangular frame FL, and the size of the character is a size not exceeding that of the input quadrangular frame FL.

However, in the irritated state, the distracted state, and the angered state, according to these emotional states, the central position of the character is displaced from the central position of the input quadrangular frame FL, and the character extends off the input quadrangular frame FL or becomes larger than the input quadrangular frame FL. Accordingly, the emotion estimation database constructing device calculates, according to each of the emotional states, a range of displacement of the character from the central position, a change range of the size of the character, and the like as a characteristic pattern at the time of character writing as an example of the pen state information.

As the “characteristic pattern at the time of character writing,” not only the characteristic pattern of one character but also a characteristic pattern between a plurality of characters at a time of successively writing the plurality of characters may be calculated. In the present embodiment, the name of each driver which name is set as the above-described specific pattern is used as the “characteristic pattern at the time of character writing.”

The emotion estimation database constructing device stores information obtained by associating information regarding each of the emotional states of the driver 2 and the range information of each of the plurality of kinds of pen state information with each other in the emotion estimation database 502 in association with the pen ID of the electronic pen 20.

FIG. 10 depicts an example of the contents stored in the emotion estimation database 502 at this time. As depicted in this FIG. 10, the respective emotional states of the “relaxed state,” the “concentrated state,” the “irritated state,” the “distracted state,” and the “angered state” are stored in association with the pen ID of the electronic pen 20, and information regarding a possible range (existence range) of each piece of pen state information in each emotional state is stored in association with each emotional state.

In the example of FIG. 10, “coordinate indication variations,” the “pen pressure,” the “inclination,” the “height position,” “positional variations at a hover position,” and the like at a time of writing input are stored as the pen state information. The range of displacement of the character from the central position and the change range of the size of the character described with reference to the foregoing FIG. 9 are stored as the coordinate indication variations. Incidentally, FIG. 10 schematically depicts the displacement range and the change range, and quadrangular frames in FIG. 10 correspond to the quadrangular frame FL in FIG. 9 described above.

Incidentally, in the case of the “coordinate indication variations,” the speed or acceleration of the variations changes mildly or changes sharply according to the emotional states. Thus, these pieces of information may also be stored in the emotion estimation database 502.

The “pen pressure” is expected to be different in value when the user performs input on the sensor by the electronic pen 20 in each of the emotional states. Thus, a possible pen pressure range (for example, Pra1 to Prb1 or the like) in each of the emotional states is stored. For example, the pen pressure in the concentrated state is higher than the pen pressure in the relaxed state, and the pen pressure range of the pen pressure in the concentrated state is narrower than that of the pen pressure in the relaxed state. In addition, in the irritated state, the pen pressure varies, and therefore the pen pressure range is widened. In the angered state, the pen pressure is increased. Incidentally, not only the range of the pen pressure but also an average value may be stored. In addition, temporal changes in the pen pressure may also be stored in the emotion estimation database 502. For example, a degree of temporal change in the pen pressure is small in the “relaxed state” and the “concentrated state,” while the degree of temporal change in the pen pressure is large in the “irritated state” and the “distracted state.”

As for the “inclination,” an angle that the electronic pen 20 forms with the sensor surface (input surface) changes according to the emotional state of the user. Thus, an inclination range (for example, SLa1 to SLb2) is stored in the emotion estimation database 502 according to each of the emotional states. Incidentally, also in this case, temporal changes in the inclination may also be stored in the emotion estimation database 502. For example, a degree of temporal change in the inclination is small in the “relaxed state” and the “concentrated state,” while the degree of temporal change in the inclination is large in the “irritated state” and the “distracted state.” Incidentally, the inclination when the pen pressure is zero and the electronic pen 20 is in a hovering state and the inclination when the pen pressure is higher than zero and the electronic pen 20 is in contact with the sensor may be stored in the emotion estimation database 502 separately from each other.

As for the “height position,” the emotion estimation database 502 stores range information (for example, Ha1 to Hb1) of possible height positions (height positions in a direction orthogonal to the sensor surface) when the electronic pen 20 is in a hovering state in a standby state before the driver 2 in the above-described various kinds of emotional states performs an indication input (writing input) while holding the electronic pen 20.

The “positional variations at the hover position” are range information (for example, Ara1 to Arb1) of possible changes in the coordinate position on the sensor surface at the time of each of the emotional states in the standby state before the driver 2 performs an indication input (writing input) while holding the electronic pen 20. Specifically, for example, the “positional variations at the hover position” are small when the driver 2 is in the “concentrated state,” while the “positional variations at the hover position” are large when the driver 2 is in the “irritated state” and the “distracted state.” Incidentally, also in this case, temporal changes in the “positional variations at the hover position” may also be stored in the emotion estimation database 502.

In the emotion server device 50, the wireless communication circuit 503 transfers the received emotional state estimation request to the emotional state estimation request receiving circuit 504. The emotional state estimation request receiving circuit 504 extracts the pen ID and the writing trajectory information included in the received emotional state estimation request, and passes the pen ID and the writing trajectory information to the emotional state estimating circuit 505. The emotional state estimating circuit 505 searches the emotion estimation database 502 on the basis of the received pen ID and each piece of pen state information of the received writing trajectory information, estimates a corresponding emotional state, and passes the emotional state as a result of the estimation to the provision information generating circuit 506.

The provision information generating circuit 506 generates provision information including information for notifying the emotional state as the estimation result received, and transmits the provision information to the management server device 40 that has made the emotional state estimation request through the wireless communication circuit 503.

Incidentally, in the configuration example of the emotion server device 50 in FIG. 8, the processing of the emotional state estimation request receiving circuit 504, the emotional state estimating circuit 505, and the provision information generating circuit 506 may be configured as software functions processed by the control circuit 501 according to a program stored in an internal memory.

An example of main processing operation of the tablet terminal 10 and the management server device 40 of the operator state determining system according to the foregoing embodiment will next be described in the following.

### <Example of Processing Operation of Tablet Terminal 10>

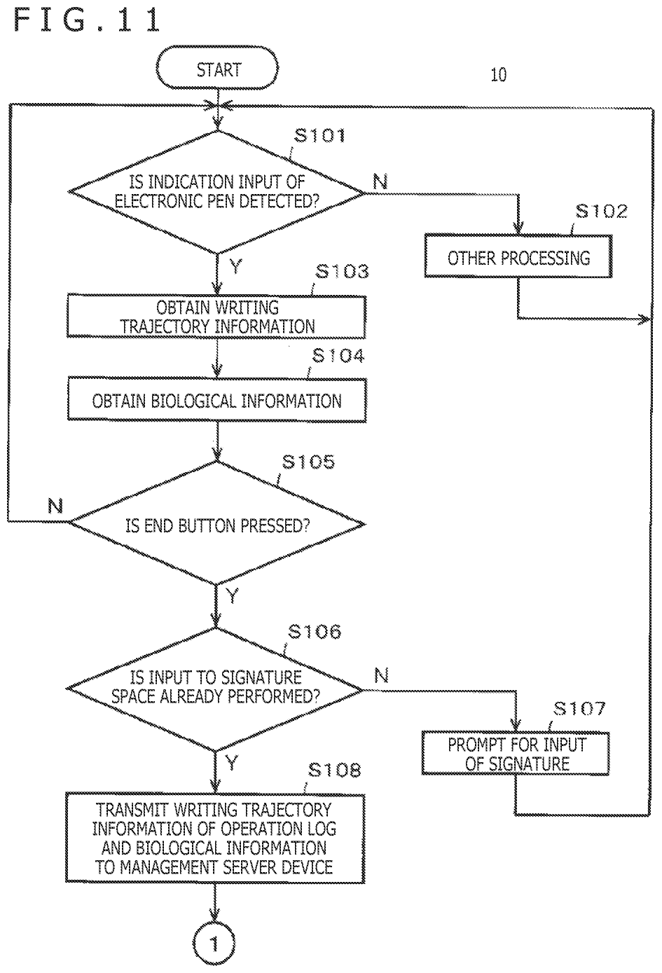

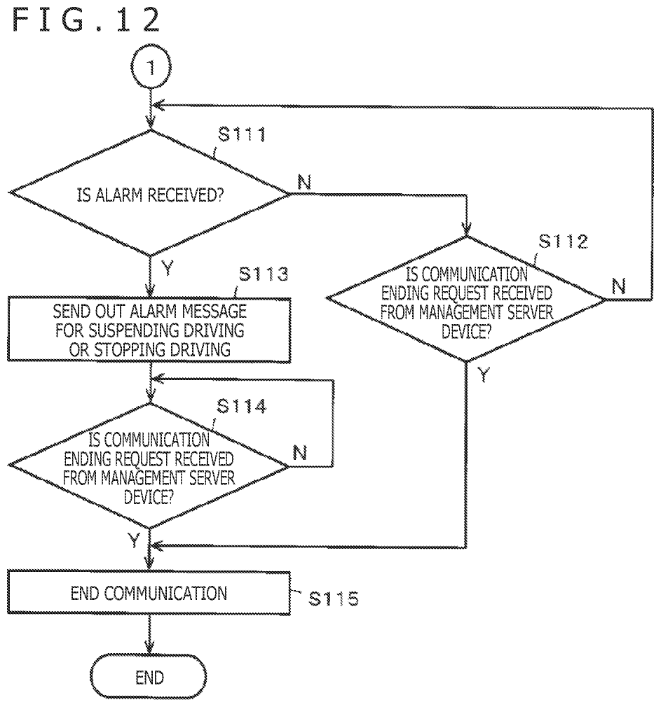

FIG. 11 and FIG. 12 as a continuation from FIG. 11 are a flowchart of assistance in explaining an example of processing operation of the tablet terminal 10.

The processing control circuit 128 of the tablet terminal 10 monitors whether or not an indication input by the electronic pen 20 is detected (S101). When the processing control circuit 128 determines that no indication input by the electronic pen 20 is detected, the processing control circuit 128 performs other processing (S102). After ending the other processing, the processing control circuit 128 returns the processing to S101.

When the processing control circuit 128 determines at S101 that an indication input by the electronic pen 20 is detected, the processing control circuit 128 obtains writing trajectory information including information obtained by adding time information to information regarding the detected indicated position, pen pressure information, information regarding the inclination, and information regarding the height position, associates the writing trajectory information with the pen ID, and supplies the resulting writing trajectory information to the controller circuit 130 (S103).

The controller circuit 130 sends a biological information obtainment request to the smart watch 30, obtains biological information sent from the smart watch 30 in response to the obtainment request, and retains the biological information in association with the pen ID together with the writing trajectory information obtained from the processing control circuit 128 (S104).

Next, the controller circuit 130 determines whether or not the end button 16 is pressed (S105). When the controller circuit 130 determines that the end button 16 is not pressed, the controller circuit 130 returns the processing to S101.

In addition, when the controller circuit 130 determines at S105 that the end button 16 is pressed, the controller circuit 130 analyzes the writing trajectory information, and determines whether or not a writing input of a signature is already performed in the signature space (S106). Then, when the controller circuit 130 determines at S106 that no writing input of the signature is performed in the signature space, the controller circuit 130 sends out a voice message prompting for the input of the signature through the speaker 11sp (S107), and thereafter returns the processing to S101.

In addition, when the controller circuit 130 determines at S106 that the writing input of the signature is already performed in the signature space, the controller circuit 130 transmits information obtained by associating the retained writing trajectory information of the operation log and the retained biological information with the pen ID to the management server device 40 through the wireless communication circuit 133 and the communication network 5 (S108).

Next, the controller circuit 130 determines whether or not an alarm is received from the management server device 40 (S111 in FIG. 12). When the controller circuit 130 determines that no alarm is received, the controller circuit 130 determines whether or not a communication ending request from the management server device 40 is received (S112). When the controller circuit 130 then determines at S112 that no communication ending request is received, the controller circuit 130 returns the processing to S111. When the controller circuit 130 determines that a communication ending request is received, the controller circuit 130 performs communication ending processing (S115), and ends this processing routine.

In addition, when the controller circuit 130 determines at S111 that an alarm is received from the management server device 40, the controller circuit 130 emits a corresponding alarm sound through the speaker 11sp (S113).

Then, the controller circuit 130 determines whether or not a communication ending request from the management server device 40 is received (S114). When the controller circuit 130 then determines at S114 that no communication ending request is received, the controller circuit 130 returns the processing to S114. When the controller circuit 130 determines that a communication ending request is received, the controller circuit 130 performs communication ending processing (S115), and ends this processing routine.

### <Example of Processing Operation of Management Server Device 40>

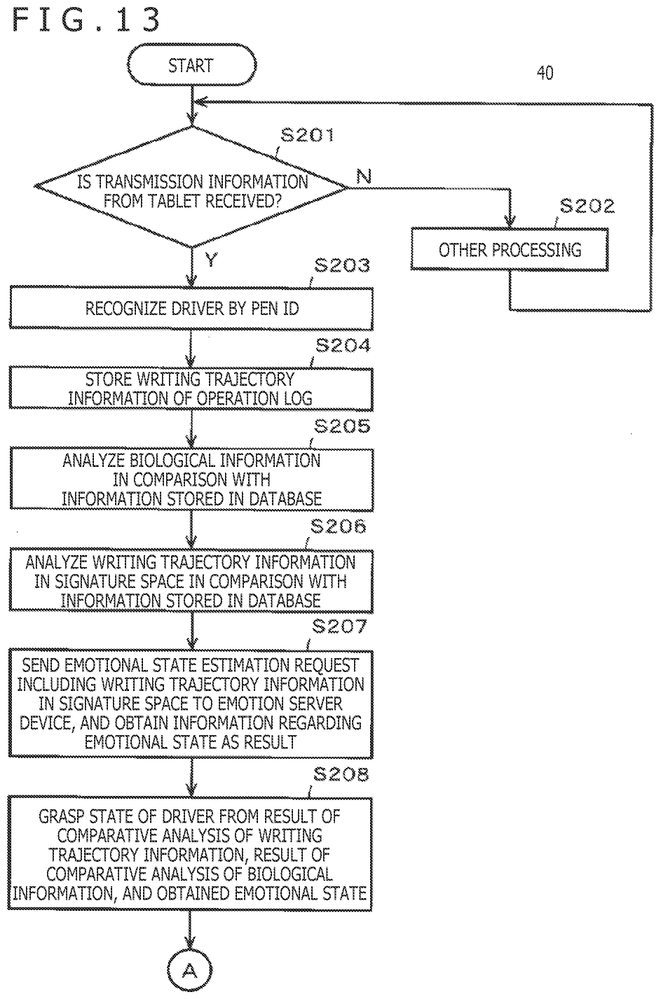

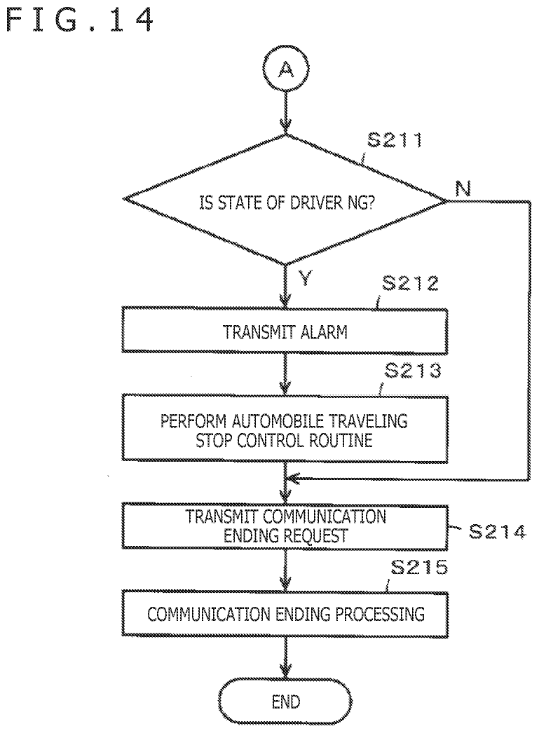

FIG. 13 and FIG. 14 as a continuation from FIG. 13 are a flowchart of assistance in explaining an example of processing operation of the management server device 40. In the following description, operation of each of the flowchart of FIG. 13 and FIG. 14 will be described as operation in a case where the control circuit 401 implements, as software processing, functions of the information reception processing circuit 403, the biological information analyzing circuit 408, the writing trajectory information analyzing circuit 409, the emotional state information obtaining circuit 410, the state determining circuit 411, the driver transmission information generating circuit 412, and the automobile control information generating circuit 413.

The control circuit 401 monitors for reception of transmission information from the tablet terminal 10 (S201). When the control circuit 401 determines that no transmission information is received from the tablet terminal 10, the control circuit 401 performs other processing (S202), and thereafter returns the processing to S201.

When the control circuit 401 determines at S201 that transmission information from the tablet terminal 10 is received, the control circuit 401 obtains a driver ID by referring to the specific pattern writing trajectory information database 405 on the basis of a pen ID included in the received information (S203). Then, the control circuit 401 stores the writing trajectory information of an operation log in the operation log memory 407 in association with the driver ID and the pen ID (S204).

Next, the control circuit 401 reads biological information associated with the driver ID obtained at S203 within the biological information stored in the biological information database 404, analyzes biological information received through the wireless communication circuit 402 in comparison with the read biological information, and thereby checks whether the physical condition state of the driver 2 identified by the driver ID is an inappropriate state for continuing driving (S205).

Next, the control circuit 401 extracts the writing trajectory information of a signature in the signature space from the writing trajectory information of the operation log, and reads the writing trajectory information of the signature of the driver 2 corresponding to the driver ID obtained by using the pen ID at S203 from the writing trajectory information of signatures which writing trajectory information is stored in the specific pattern writing trajectory information database 405. Then, the control circuit 401 analyzes the extracted writing trajectory information of the signature in comparison with the writing trajectory information of the signature which writing trajectory information is read from the specific pattern writing trajectory information database 405, and thereby checks whether the mental state of the driver 2 identified by the driver ID is an inappropriate state for continuing driving (S206).

Next, the control circuit 401 sends an emotional state estimation request including the extracted writing trajectory information of the signature to the emotion server device 50, and obtains information regarding an estimated emotional state as a reply to the emotional state estimation request from the emotion server device 50 (S207).

Next, the control circuit 401 grasps the physical condition state and the mental state of the driver who has transmitted the writing trajectory information of the operation log from a result of the analysis of the biological information which result is obtained at S205, a result of the analysis of the specific pattern writing trajectory information, and a result of estimation of the emotional state which result is obtained from the emotion server device 50 (S208).

Then, the control circuit 401 determines whether the state of the driver 2 is an inappropriate state for driving from the physical condition state and the mental state of the driver 2, the physical condition state and the mental state being grasped at S208 (S211 in FIG. 14). When the control circuit 401 determines that the state of the driver 2 is not the inappropriate state, the control circuit 401 transmits a communication ending request to the tablet terminal 10 of the driver 2 (S214), performs processing of ending wireless communication (S215), and ends this processing routine.

In addition, when the control circuit 401 determines at S211 that the state of the driver 2 is an inappropriate state for driving, the control circuit 401 in the present example generates an alarm including a voice message, and transmits the alarm to the tablet terminal 10 of the driver 2 (S212).

Next, the control circuit 401 performs an automobile stop control routine that suspends or stops traveling of the automobile 1 of the driver 2 in an inappropriate state for driving (S213). In the automobile stop control routine, the control circuit 401 controls the automobile by obtaining connection information for connection to the automobile 1 of the driver 2 in an inappropriate state for driving, forming a wireless communication path to the automobile 1, and transmitting control information for suspending or stopping traveling of the automobile 1 to the automobile 1.

After S213, the control circuit 401 transmits a communication ending request to the tablet terminal 10 of the driver 2 (S214), performs processing of ending wireless communication (S215), and ends this processing routine.

As described above, the operator state determining system according to the first embodiment checks not only for an abnormality in the physical condition state on the basis of the biological information of the driver 2 but also whether or not the mental state of the driver 2 is an inappropriate state for driving from the writing trajectory information of the operation log, and issues an alarm to the driver to make the driver 2 refrain from driving the automobile 1 when the mental state of the driver 2 is the inappropriate state. Hence, the occurrence of a traffic accident can be prevented.

Furthermore, the foregoing embodiment performs control so as to suspend or stop traveling of the automobile as a target for driving by the driver 2 by a control signal from the management server device 40 when the state (the physical condition state and/or the mental state) of the driver 2 is an inappropriate state for driving. Thus, the occurrence of a traffic accident can be prevented.

In addition, because the mental state of the driver 2 is checked by using the writing trajectory information of the operation log, there are effects in that it suffices for the driver 2 merely to perform writing input to the operation log and no special burden needs to be imposed on the driver 2 to check the mental state of the driver 2.

Moreover, the foregoing first embodiment does not simply determine whether or not the mental state of the driver 2 is an appropriate state for driving only from deviation from information when the mental state of the driver 2 is appropriate as registered specific pattern writing trajectory information, but refers to also a result of grasping the emotional state estimated from the writing trajectory information by using the emotion server device 50. Thus, there is also an effect of being able to determine more accurately whether or not the mental state of the driver 2 is an appropriate state for driving.

Incidentally, while the emotion estimation database of the emotion server device 50 stores correspondences between the pen state information generated from the specific pattern writing trajectory information of each driver 2 and the emotional states in the foregoing embodiment, the writing trajectory information of another writing pattern rather than the specific pattern writing trajectory information may be used in constructing the emotion estimation database.

In addition, the emotion server device 50 may include an emotion estimation database constructed from the emotional states of a large number of unspecified people and the pen state information generated from the writing trajectory information at times of the respective emotional states. In this case, the accuracy of estimating the emotional states in the emotion server device 50 is improved when the emotional states of a large number of unspecified people and the pen state information generated from the writing trajectory information at the times of the respective emotional states are collected in large quantities as generally-called big data.

In addition, while the tablet terminal 10 has a function of wirelessly communicating with the management server device 40 in the foregoing first embodiment, a configuration may be adopted in which a wireless communication terminal device having a function of wirelessly communicating with the management server device 40 is provided separately, and a connection between the tablet terminal 10 and the wireless communication terminal device is established by wire or by short-range wireless communication. A portable communication device such, for example, as a smart phone can be used as the wireless communication terminal device in that case, and an alarm from the management server device 40 may be displayed on the display screen of the portable communication device and notified as a voice message.

In addition, while the emotion server device 50 is configured separately from the management server device 40 in the foregoing first embodiment, a configuration can also be adopted in which the functions of the emotion server device 50 are integrated by being provided to the management server device 40.

In addition, the foregoing embodiment uses, as an example of the writing trajectory information obtaining means, the electronic pen 20 having the function of the ballpoint pen as a writing instrument and the tablet terminal 10 configured to allow writing to the operation log paper 6 by the electronic pen 20 and to be able to generate the writing trajectory information as electronic data. However, the writing trajectory information obtaining means may be an electronic pen and a tablet device of an ordinary configuration that does not allow writing to the operation log paper 6 but is able to obtain the writing trajectory information as electronic data.

In addition, while heartbeat, body temperature, and perspiration are illustrated as the biological information of the driver in the foregoing example, other biological information, that is, a blood pressure, a blood sugar, a pulse, or the like may be used, and further combinations thereof may be used.

### Second Embodiment

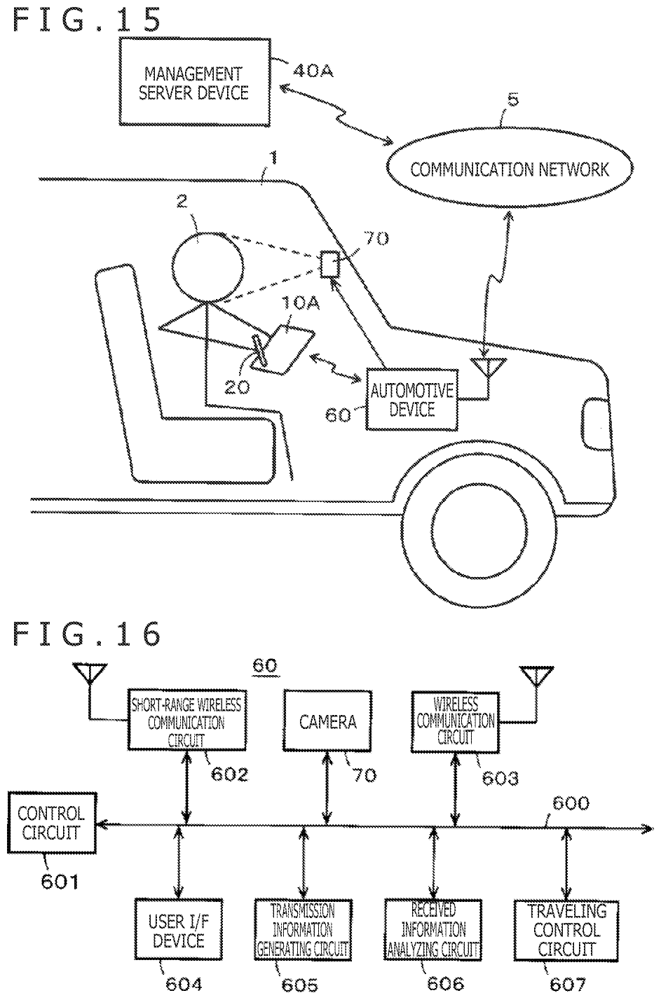

A second embodiment is also an example of the operator state determining system operated by the taxi company depicted in FIG. 1, and is a modification of the foregoing first embodiment. FIG. 15 depicts a diagram of assistance in explaining an outline of the operator state determining system according to the second embodiment.

In the foregoing first embodiment, the heartbeat, the body temperature, perspiration, or the like is collected as the biological information of the driver 2, and the driver 2 wears the smart watch 30 including a heartbeat sensor and a body temperature sensor in order to obtain the biological information. On the other hand, in the second embodiment, a face image of the driver 2 is collected as the biological information of the driver 2. In this case, the face image is obtained as a color image so that a facial color, a lip color, or the like can be recognized.

In addition, in the foregoing first embodiment, the tablet terminal 10 of the driver 2 collects not only the writing trajectory information but also the biological information, and includes wireless communication means for transmitting the writing trajectory information and the biological information to the management server device 40. On the other hand, in the second embodiment, the automobile 1 used for service as a taxi is provided with an automotive device 60 capable of wireless communication with a management server device 40A.

Incidentally, the management server device 40A according to the second embodiment is configured as a management server device in which the functions of the management server device 40 and the emotion server device 50 in the first embodiment are integrated with each other. Then, though not depicted, the management server device 40A includes a biological information database in which information of a color face image of each driver 2 in a good physical condition state is stored in association with the driver ID. The face image includes face images photographed not only from a front but also from a plurality of directions, that is, from a left and a right side. Then, the management server device 40A includes not only the specific pattern writing trajectory information database but also the emotion estimation database.

A biological information analyzing circuit of the management server device 40A according to the second embodiment detects an abnormality in the physical condition such as looking pale, or purple lips by comparing a face image of the driver 2 photographed by a camera 70 at a time of writing input of the writing trajectory information of an operation log with the face image of the driver 2 which face image is stored in the database in advance. In addition, the expression of the driver 2 is analyzed from the face image of the driver 2, and a facial expression at a time of pain, a facial expression at a time of an extreme fatigue, and the like are also detected.

Then, in the second embodiment, a camera 70 for photographing the face image of the driver 2 riding in a driver's seat is provided in a state of being connected to the automotive device 60 of the automobile 1. The camera 70 is connected to the automotive device 60 by wire or by radio. The automotive device 60 controls operation of the camera 70 and captures the face image information of the driver 2 photographed by the camera 70.

In addition, in the second embodiment, a tablet terminal 10A used by each driver 2 does not include the controller circuit 130, but instead includes a short-range wireless communication circuit that wirelessly connects to a short-range wireless communication circuit provided to the automotive device 60. Other configurations of the tablet terminal 10A are similar to those of the tablet terminal 10 in the first embodiment. Incidentally, also in the second embodiment, each of the drivers 2 performs writing input to the tablet terminal 10A by the electronic pen 20 used in the foregoing first embodiment.

The configuration and processing operation of the tablet terminal 10A are similar to those of the tablet terminal 10 except for the part of the controller circuit 130, and therefore description thereof will be omitted here together with the operation of the electronic pen 20. In addition, the configuration and processing operation of the management server device 40A are similar to those of the management server device 40 in the foregoing first embodiment only with a difference in that the management server device 40A includes constituent parts of the emotion server device 50 and also performs the functional processing operation of the emotion server device 50. Thus, description of the configuration and processing operation of the management server device 40A will be omitted here.

Accordingly, in the second embodiment, description will be made of a configuration example and processing operation of the automotive device 60.

### <Example of Hardware Configuration of Automotive Device 60>

FIG. 16 is a block diagram depicting an example of a hardware configuration of the automotive device 60 in the second embodiment. In the automotive device 60 in the example of FIG. 16, a control circuit 601 constituted by a computer (e.g., including a processor and a memory storing instructions that, when executed by the processor, cause the control circuit 601 to perform the functions of the control circuit 601 described herein) is connected with a short-range wireless communication circuit 602, a wireless communication circuit 603, a user interface device 604, a transmission information generating circuit 605, a received information analyzing circuit 606, and a traveling control circuit 607 through a system bus 600, and the camera 70 is further connected to the system bus 600.

The short-range wireless communication circuit 602 is for communicating with the short-range wireless communication circuit provided to the tablet terminal 10. The short-range wireless communication circuit 602 in the present example has a configuration of a wireless communication circuit of the Bluetooth (registered trademark) standard. The wireless communication circuit 603 is for communicating with the management server device 40A through the communication network 5.

Though not depicted, the user interface device 604 includes a display and a speaker. The user interface device 604 displays an alarm from the management server device 40A and emits the alarm by sound.

The transmission information generating circuit 605 generates transmission information from writing trajectory information (provided with a pen ID) from the tablet terminal 10A which writing trajectory information is obtained through the short-range wireless communication circuit 602 and face image information of the driver 2 which face image information is obtained from the camera 70. The transmission information generating circuit 605 transmits the generated transmission information to the management server device 40A through the wireless communication circuit 603.

The received information analyzing circuit 606 analyzes information sent from the management server device 40A and performs processing according to a result of the analysis. When the received information analyzing circuit 606 determines that an alarm is received from the management server device 40A, the received information analyzing circuit 606 controls the user interface device 604 so as to display the received alarm on the display and emit an alarm sound from the speaker, and sends a traveling stop control signal to the traveling control circuit 607.

When the traveling control circuit 607 receives the traveling stop control signal from the received information analyzing circuit 606, the traveling control circuit 607 performs control so as to suspend or stop traveling of the own vehicle.

Incidentally, in the configuration example of the automotive device 60 in FIG. 16, the processing of the transmission information generating circuit 605, the received information analyzing circuit 606, and the traveling control circuit 607 may be configured as software functions processed by the control circuit 601 according to a program stored in an internal memory.

### <Example of Processing Operation of Automotive Device 60>

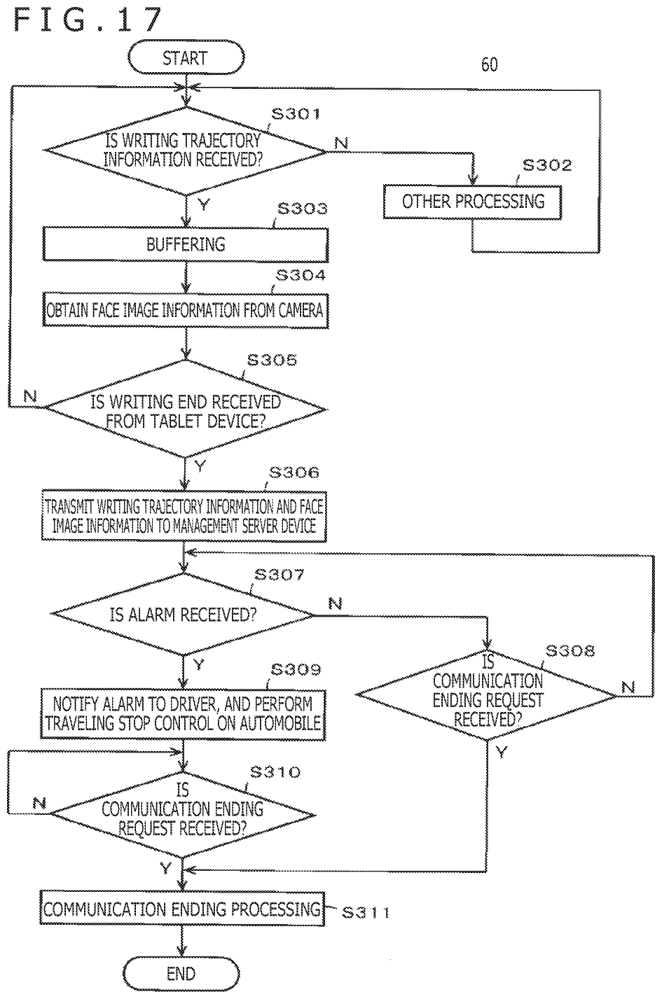

FIG. 17 is a flowchart of assistance in explaining an example of processing operation of the automotive device 60. In the following description, processing of each of the flowchart of FIG. 17 will be described as processing in a case where the control circuit 601 configures the processing of the transmission information generating circuit 605, the received information analyzing circuit 606, and the traveling control circuit 607 as software functions processed according to the program stored in the internal memory.

The control circuit 601 determines whether or not writing trajectory information from the tablet terminal 10 is received by the short-range wireless communication circuit 602 (S301). When the control circuit 601 determines that no writing trajectory information is received, the control circuit 601 performs other processing (S302), and thereafter returns the processing to S301.

When the control circuit 601 determines at S301 that writing trajectory information is received, the control circuit 601 temporarily stores and retains the received writing trajectory information (provided with time information) in a buffer memory in association with the pen ID (S303). Next, the control circuit 601 obtains photographing information of a face image of the driver 2 photographed by the camera 70, and retains the photographing information in the buffer memory in association with the writing trajectory information (S304).

Then, the control circuit 601 determines whether or not a writing end is received from the tablet terminal 10 (S305). When the control circuit 601 determines that no writing end is received, the control circuit 601 returns the processing to S301, and repeats the processing from S301 on down.

In addition, when the control circuit 601 determines at S305 that a writing end is received from the tablet terminal 10, the control circuit 601 transmits information obtained by associating the writing trajectory information of the operation log which writing trajectory information is stored in the buffer memory and the information of the face image of the driver 2 as biological information with the pen ID to the management server device 40A through the wireless communication circuit 603 and through the communication network 5 (S306).

Then, the control circuit 601 determines whether or not an alarm is received from the management server device 40A (S307). When the control circuit 601 determines that no alarm is received, the control circuit 601 determines whether or not a communication ending request is received (S308). When the control circuit 601 determines in this S308 that no communication ending request is received, the control circuit 601 returns the processing to S307, and repeats the processing from S307 on down. When the control circuit 601 determines at S308 that a communication ending request is received, the control circuit 601 performs processing of ending wireless communication processing with the management server device 40A (S311), and ends this processing routine.

In addition, when the control circuit 601 determines at S307 that an alarm is received, the control circuit 601 displays the alarm on the display of the user interface device 604 and emits an alarm sound by the speaker, thereby warning the driver 2 to suspend or stop driving, and performs control so as to suspend or stop traveling of the own vehicle (S309). Then, the control circuit 601 waits to receive a communication ending request from the management server device 40A (S310). When the control circuit 601 determines that a communication ending request is received, the control circuit 601 performs processing of disconnecting a wireless communication path to the management server device 40A (S311). Then, the control circuit 601 ends this processing routine.

The operator state determining system according to the second embodiment provides actions and effects similar to those of the foregoing first embodiment, and provides an effect in that it suffices for the driver 2 merely to perform an operation of simple writing input of the writing trajectory information of an operation log without a need for wearing a smart watch. In addition, the tablet terminal 10 neither needs to have a function of collecting the biological information nor needs to have a function of wireless communication with the management server device 40A. An effect is thus produced in that the tablet terminal 10 can be of a simple configuration merely having a function of generating the writing trajectory information.

### Other Embodiments or Modifications

It is to be noted that while the electronic pen and the position detecting device of the tablet terminal in the foregoing embodiments are of an electromagnetic induction type, the electronic pen and the position detecting device are not limited to this. It is needless to say that an electronic pen and a position detecting device of an active capacitive type, for example, can be used.

The biological information collected in the foregoing first embodiment is of course not limited to the heartbeat and the body temperature.

In addition, the first embodiment and the second embodiment described above represent a case where the operator is a driver, and the automobile as an operation target is a taxi. However, it is needless to say that the first embodiment and the second embodiment are not limited to this either. For example, the operation target may be a truck that transports a load, and the operator may be a driver of the truck, or the operation target may be a bus, and the operator may be a driver of the bus. In addition, the operation target is not limited to the automobile, but may be a motorcycle or a bicycle.

In addition, the automobile is not limited to those traveling on land, but may be a vehicle for use both in water and on land or a vehicle for use both in air and on land. In addition, the operation target may be a vessel or an aircraft rather than the automobile.

It is to be noted that the embodiments of the present disclosure are not limited to the foregoing embodiments, and that various changes can be made without departing from the spirit of the present disclosure

What is claimed is:

1. An operator state determining system comprising:
an information collecting device which, in operation, collects information used to determine whether an operator who operates an operation target device is in an inappropriate state for an operation; and
a determining device which, in operation, determines whether the operator is in the inappropriate state for the operation,
the information collecting device including:
a biological information obtaining device which, in operation, obtains biological information of the operator,
a writing trajectory information obtaining device which, in operation, receives writing input by the operator, and obtains writing trajectory information corresponding to the writing input, and
a transmission circuit which, in operation, transmits the biological information obtained by the biological information obtaining device and the writing trajectory information obtained by the writing trajectory information obtaining device to the determining device,
the determining device including:
a reception circuit which, in operation, receives the biological information and the writing trajectory information transmitted from the information collecting device, and
a state determining circuit which, in operation, determines whether the operator is in the inappropriate state for the operation based on the biological information and the writing trajectory information received by the reception circuit.

2. The operator state determining system according to claim 1, wherein:
the writing trajectory information obtaining device includes an electronic pen used by the operator to perform the writing input and a position detecting device which, in operation, detects a position indicated by the electronic pen and outputs the writing trajectory information.

3. The operator state determining system according to claim 2, wherein:
the electronic pen includes a writing instrument which, in operation, performs writing on paper, and
the position detecting device, in operation, detects the position indicated by the electronic pen when the writing is performed on the paper by the writing instrument of the electronic pen.

4. The operator state determining system according to claim 2, wherein:
the electronic pen includes a pen pressure detector, and includes a transmission circuit, which, in operation, transmits pen pressure information detected by the pen pressure detector to the position detecting device, and
the writing trajectory information output from the position detecting device includes coordinate information of the position indicated by the electronic pen and the pen pressure information.

5. The operator state determining system according to claim 2, wherein:
the position detecting device, in operation, obtains time information when the position indicated by the electronic pen is detected, and the writing trajectory information output from the position detecting device includes coordinate information of the position indicated by the electronic pen and the time information corresponding to the coordinate information of the indicated position.

6. The operator state determining system according to claim 2, wherein:
the electronic pen includes a pen pressure detector, and includes a transmission circuit which, in operation, transmits pen pressure information detected by the pen pressure detector to the position detecting device, and
the position detecting device, in operation, obtains time information when the position indicated by the electronic pen is detected, and the writing trajectory information output from the position detecting device includes coordinate information of the position indicated by the electronic pen, the time information corresponding to the coordinate information of the indicated position, and the pen pressure information.

7. The operator state determining system according to claim 1, wherein:
the determining device includes a storage device which, in operation, stores specific pattern writing trajectory information when the operator performs the writing input of a writing trajectory in a predetermined specific pattern,
the writing trajectory information obtaining device, in operation, obtains writing trajectory information of the specific pattern input by writing by the operator, and
the state determining circuit, in operation, makes the determination by using the writing trajectory information of the specific pattern, the writing trajectory information being included in the information received from the information collecting device, and the specific pattern writing trajectory information stored by the storage device.

8. The operator state determining system according to claim 2, wherein:
the electronic pen, in operation, transmits identification information to the position detecting device,
the position detecting device, in operation, obtains the writing trajectory information based on the position indicated by the electronic pen in association with the identification information,
the transmission circuit which, in operation, transmits the writing trajectory information to the determining device in association with the identification information,
the determining device includes a storage device which, in operation, stores specific pattern writing trajectory information when the operator performs the writing input of a writing trajectory in a predetermined specific pattern in association with the identification information,
the writing trajectory information obtaining device, in operation, obtains writing trajectory information of the specific pattern input by writing by the operator in association with the identification information, and
the state determining circuit, in operation, makes the determination by using the writing trajectory information of the specific pattern, the writing trajectory information being included in the information received from the information collecting device, and the specific pattern writing trajectory information stored by the storage device, the writing trajectory information and the specific pattern writing trajectory information being assigned identical identification information.

9. The operator state determining system according to claim 1, wherein;
the biological information of the operator includes one of heartbeat, blood pressure, blood sugar, body temperature, and perspiration of the operator or a combination of the heartbeat, the blood pressure, the blood sugar, the body temperature, and the perspiration of the operator, and the biological information obtaining device is worn by an arm of the operator.

10. The operator state determining system according to claim 1, wherein:
the biological information obtaining device a camera which, in operation, photographs and obtains a face image of the operator as the biological information.

11. The operator state determining system according to claim 1, wherein;
the determining device includes an emotional state estimating circuit which, in operation, estimates an emotional state of the operator at a time of the writing input from the writing trajectory information from the information collecting device, and the state determining circuit, in operation, determines whether the operator is in the inappropriate state for the operation using information regarding the emotional state of the operator estimated by the emotion estimating circuit.

12. The operator state determining system according to claim 11, wherein:
the determining device includes an emotional state database configured to store each of a plurality of kinds of emotional states of a writer at times of performing writing input and writing trajectory information of the writing input at a time of each of the plurality of kinds of emotional states in association with each other.

13. The operator state determining system according to claim 1, wherein:
the information collecting device and the determining device are wirelessly connected to each other through a network.

14. The operator state determining system according to claim 13, wherein:
the network is connected with an emotion estimating device which, in operation, estimates an emotion of the operator at a time of the writing input from the writing trajectory information from the information collecting device, and
the state determining circuit of the determining device determines whether the operator is in the inappropriate state for the operation using information regarding the emotion of the operator estimated by the emotion estimating device.

15. The operator state determining system according to claim 1, wherein:
the determining device is wirelessly connected to the operation target device through a network, and the determining device, in operation, supplies a control signal that stops operation of the operation target device through the network when the state determining means determines that the operator is in the inappropriate state for the operation.

16. The operator state determining system according to claim 1, wherein:
the determining device is wirelessly connected to a communication device possessed by the operator through a network, and the determining device, in operation, transmits an alarm to the communication device through the network when the state determining circuit determines that the operator is in the inappropriate state for the operation.
